(19) United States
(12) Patent Application Publication
(10) Pub. No.: US 2012/0134979 A1
(43) Pub. Date: May 31, 2012

(54) METHODS AND COMPOSITIONS FOR THE TREATMENT OF SICKLE CELL DISEASE

(76) Inventors: Yang Xia, Bellaire, TX (US); Rodney E. Kellems, Bellaire, TX (US); Yujin Zhang, Houston, TX (US); Harinder S. Juneja, Sugarland, TX (US)

(21) Appl. No.: 13/302,985

(22) Filed: Nov. 22, 2011

Related U.S. Application Data
(60) Provisional application No. 61/415,944, filed on Nov. 22, 2010.

(51) Int. Cl. A61K 38/50; A61P 7/06; A61K 31/519; A61K 31/52; A61K 31/522; A61K 31/191

(52) U.S. Cl. 424/94.6; 514/263.34; 514/557; 514/267; 514/263.4

(57) ABSTRACT

Presented are mechanism based compositions and methods for treatment of SCD and SCD associated symptoms and disorders, particularly increased RBC sickling, HbS polymerization, hemolysis, tissue congestion and disruption and organ damage or failure in a mammal. The disclosed methods feature the identification of the heretofore unknown role of adenosine levels and signaling in the development of SCD and SCD associated symptoms and disorders. This discovery has lead to the identification of compositions for use as therapies for SCD and SCD associated disorders and symptoms in a mammal.

This application claims the benefit under 35 U.S.C. §119 of prior-filed, co-pending provisional application 61/415,944, filed Nov. 22, 2010.

### STATEMENT REGARDING FEDERALLY SPONSORED RESEARCH OR DEVELOPMENT

This invention was made with government support under Grant Nos. R01 DK046207; DK077748 and DK083559 awarded by the National Institutes of Health. The government has certain rights in the invention.

### TECHNICAL FIELD

This disclosure generally relates to compositions and methods for treatment of sickle cell disease (SCD) and SCD associated symptoms and disorders in mammals, such symptoms include, but are not limited to, the sickling of erythrocytes, oxygen release, increased hemoglobin (HbS) polymerization, hemolysis, tissue congestion and disruption and organ damage or failure. The disclosed methods feature the identification of the heretofore unknown role of adenosine levels and signaling in the development of SCD and SCD associated symptoms and disorders. This discovery has lead to the identification that compositions that act as A_{2B}R antagonists can be used, to reduce the sickling of erythrocytes and thus as therapies for SCD and SCD associated symptoms and disorders (collectively, “sickle cell disease” or “SCD”).

### BACKGROUND

This section of this document introduces various aspects of the art that may be related to various aspects of the presently described and/or claimed methods. It provides background information to facilitate a better understanding of various aspects of the present invention. As the section's title implies, this is a discussion of “related” art. That such art is related in no way implies that it is also “prior” art. The related art may or may not be prior art. The discussion in this section of this document is to be read in this light, and not as admissions of prior art.

Sickle-cell disease (SCD), or sickle-cell anemia (or drepanocytosis), is a life-long blood disorder characterized by red blood cells (erythrocytes: RBC) that assume an abnormal, rigid, sickle shape. Sickling decreases flexibility of RBC and results in a risk of various complications. RBC sickling occurs because of a mutation in the hemoglobin gene. SCD is an inherited disorder and SCD is an autosomal recessive disease. Although, some people who inherit one sickle cell gene and one other defective hemoglobin gene may experience a similar sickle-cell disorder.

SCD affects millions of people worldwide. It is most common in people whose families come from Africa, South or Central America (especially Panama), Caribbean islands, Mediterranean countries (such as Turkey, Greece, and Italy), India, and Saudi Arabia. One-third of all indigenous inhabitants of Sub-Saharan Africa carry the gene. In the United States, according to the National Institutes of Health, sickle cell anemia affects about 70,000 people. The disease occurs in about 1 out of every 500 African American births. Sickle cell anemia also affects Hispanic Americans. The disease occurs in 1 out of every 36,000 Hispanic American births. About 2 million Americans carry the sickle cell gene and this occurs in about 1 in 12 African Americans. Because it is an inherited disorder, SCD usually presents in childhood and as a result life expectancy is shortened.

SCD is a devastating inherited hemolytic disorder that affects RBC due to a single point mutation which results in the substitution of valine for glutamic acid at sixth position of the β-globin chain of hemoglobin (Ingram, V. M. A specific chemical difference between the globins of normal human and sickle-cell anaemia haemoglobin. Nature 178, 792-794, 1956; Madigan, C. & Malik, P. Pathophysiology and therapy for haemoglobinopathies. Part I: sickle cell disease. Expert reviews in Molecular Medicine 8, 1-23, 2006; Urbinati, F., Madigan, C. & Malik, P. Pathophysiology and therapy for haemoglobinopathies. Part II: thalassaemias. E Expert reviews in Molecular Medicine 8, 1-26, 2006).

Therapies that have been tried to address SCD and SCD related disorders include dietary cyanate using foods containing cyanide derivatives. Most people with SCD experience intensely painful episodes known as vaso-occlusive crisis. The frequency, severity, and duration of these crises, however, vary from patient to patient. Painful crisis is treated symptomatically with analgesics and pain management may require opioid administration at regular intervals until the crisis has settled. For patients experiencing a less severe crisis some can be managed using nonsteroidal anti-inflammatory drugs (NSAIDs, such as diclofenac or naproxen). Patients experiencing more severe crisis may require inpatient management for intravenous opioids often delivered using patient-controlled analgesia devices. Diphenhydramine is also frequently prescribed by doctors in order to help control any itching associated with the use of opioids (Ballas, S. K. Current issues in sickle cell pain and its management. Hematology/the Education Program of the American Society of Hematology. American Society of Hematology, 97-105, 2007).

Management of patients with acute chest crisis are as described for vaso-occlusive crisis with the addition of antibiotics (usually quinolones or macrolides), oxygen supplementation requirements increase, blood transfusion or exchange transfusion may even be indicated. Exchange transfusion involves the exchange of a significant portion of the patient's RBC for normal donor RBC, which decreases the percent of hemoglobin S present in the patient's blood.

The first approved drug for the treatment of SCD was hydroxyurea, which was shown to decrease the number and severity of attacks and to possibly increase survival. Hydroxyurea is believed to act, in part, by reactivating fetal hemoglobin production in place of the sickle hemoglobin (HbS) that causes SCD. However, hydroxyurea is a known chemotherapeutic agent, and there is some concern that long-term use may be harmful (Lanzkron, S., Haywood, C., Jr., Segal, J. B. & Dover, G. J. Hospitalization rates and costs of care of patients with sickle-cell anemia in the state of Maryland in the era of hydroxyurea. American Journal of Hematology 81, 927-932, 2006).

Children born with SCD undergo close observation by a pediatrician and require management by a hematologist to assure they remain healthy. These patients take a 1 mg dose of folic acid daily for life. From birth to five years of age, they will also have to take penicillin daily due to an immature immune system that makes them more prone to early childhood illnesses. Bone marrow transplantation has shown to be an effective way to treat children with SCD, if successful.

Various other approaches examined to prevent sickling episodes as well as other complications associated with SCD, include the use of phytochemicals such as nicosan, gene therapy and the proposed use of Senicapoc (ICA-17043, Icagen, Inc.), a Gardos (KCNN4) channel blocker.

Despite early and precise knowledge of the molecular defect associated with SCD to HbS in RBC, there remains neither a preventative approach nor mechanism-specific therapy available for the SCD patient, in spite of the long standing and unmet need. Consequently, there remains great interest in selective therapeutics based on molecular selective mechanisms.

### BRIEF DESCRIPTION OF THE FIGURES

FIG. 1. Increased adenosine leads to sickling and hemolysis in SCD transgenic (Tg) mice. (a) Representative HPLC profile showing adenosine levels in the plasma of wild type (WT) and SCD Tg mice at steady state. (b) The effect of chronic PEG-ADA treatment on adenosine levels in the plasma of WT and SCD Tg mice. c. Blood smears of SCD-Tg mice as a function PEG-ADA enzyme therapy. (d-f) Effect of PEG-ADA treatment on plasma hemoglobin (d), plasma haptoglobin (e) and plasma total bilirubin (f) in wild type (WT) and SCD Tg mice. (g) Life span of RBCs in SCD Tg mice treated with or without PEG-ADA. Values shown represent the mean±SEM. N=4-8, *p<0.05 versus WT; **p<0.05, relative to untreated SCD. ND=not-detected.

FIG. 2. In vivo effects of PEG-ADA treatment. (a) Histological analysis of lung, liver and spleen of WT and SCD Tg mice treated with PEG-ADA for 8 weeks. Tissues were obtained from WT or SCD Tg mice following 8 weeks with or without PEG-ADA treatment. The results show significant congestion, vascular damage and necrosis in the lungs, livers and spleens of SCD Tg mice relative to WT that was reduced by PEG-ADA treatment. (b-d) Semiquantitative analysis of histological changes using Image-plus Pro software in multiple tissues of the mice. (e-g) PEG-ADA enzyme therapy significantly decreased the elevated heme content in lungs, livers and spleens of SCD Tg mice. h. The microinfarction and cysts seen in renal cortex and congestion in renal medulla of SCD Tg mice were significantly decreased by 8-week PEGADA enzyme therapy. (i-j) Semiquantitative analysis of histological changes using Image-plus Pro software in mouse kidney. (h-k) PEG-ADA enzyme therapy decreased proteinuria (h) and improved tubular concentration defect (k) in SCD Tg mice. Values shown represent the mean±SEM from 4-8 animals in each group. *p<0.05 relative to WT and **p<0.05 relative to untreated SCD Tg mice.

FIG. 3. PEG-ADA treatment attenuates hypoxia/reoxygenation-induced acute sickle crisis in SCD Tg mice. (a) Level of adenosine in the plasma of SCD Tg mice was further remarkably increased in response to hypoxia/reoxygenation and PEG-ADA treatment significantly lowered its elevation. (b) PEG-ADA treatment prevented hypoxia/reoxygenation-mediated further increase in percentage of sickle cells. (c-d) PEG-ADA treatment attenuated hypoxia/reoxygenation-induced remarkable hemolysis including increased plasma Hb (c) and bilirunbin (d). e. Lung sections from SCD Tg mice were stained with an Ab against neutrophils to visualize infiltrated tissue neutrophils (brown). Bars, 10 μm. (f) Quantification of neutrophil infiltration in the lung of SCD Tg mice by Image-plus Pro software. (g) PEG-ADA treatment significantly lowered hypoxia/reoxygenation-induced increased INF-γ, IL-6, IL-1β and GM-CSF proinflammatory cytokine levels in the lung homogenates in SCD Tg mice. Data are presented as mean±SEM from 5-7 animals in each group. *p<0.05 relative to SCD Tg mice under normoxic condition and **p<0.05 relative to SCD Tg mice without treatment under hypoxia/reoxygenation condition.

FIG. 4. Adenosine functions through A_{2B}R to induce 2,3-DPG levels and subsequent sickling in SCD Tg mice. (a) Concentration of 2,3-DPG in RBCs of wild type (WT) and SCD Tg mice as a function of PEG-ADA chronic enzyme therapy. (b) In vivo measurement of percentage of oxygen saturation of Hb in controls and SCD Tg mice treated with or without PEG-ADA for 8 weeks. (c) Concentration of 2,3-DPG in RBCs of SCD Tg mice under normoxic and hypoxia/reoxygenation conditions with or without PEG-ADA pre-treatment. (d) 2,3-DPG concentrations in isolated RBCs treated with NECA in the presence or absence of theophylline. (e) 2,3-DPG concentrations in cultured RBCs isolated from WT or four adenosine receptor-deficient mice treated with or without NECA. (f) Immunostaining of A_{2B}R in RBCs from WT and A_{2B}R-deficient mice. (g) 2,3-DPG concentrations in isolated RBCs from WT and adenosine receptor-deficient mice under hypoxic conditions. (h) cAMP levels of RBCs isolated from WT mice and A_{2B}R deficient mice treated with or without NECA. (i) 2,3-DPG levels in RBCs isolated from WT mice treated with NECA in the presence or absence of the PKA specific inhibitor, H-89. (j) 2,3-DPG concentrations in RBCs of WT and SCD Tg mice treated with or without PSB1115. (k) Life span of RBCs in SCD Tg mice treated with or without PSB1115. All values are expressed as mean±SEM where *p<0.05 relative to untreated controls and **p<0.05, relative to treated or positive samples. n=5-7. ND=non-detectable.

FIG. 5. Adenosine is elevated in human SCD patients and A_{2B}R-mediated 2,-3-DPG elevation is required for hypoxia-induced human erythrocyte sickling. (a) Average adenosine levels in the plasma from SCD patients (SCD, n=12) and healthy volunteers (control, n=11). (b) 2,3-DPG concentration in RBCs isolated from normal and SCD patients. n=11 for the control and n=12 for SCD patients. (c) Changes in 2,3-DPG concentration in isolated SCD RBCs following incubation under hypoxic conditions in the absence or presence of PEG-ADA, MRS1754 (an A_{2B}R antagonist), H89 (PKA specific inhibitor) or GA (glycolic acid, a compound that promotes degradation of 2,3-DPG). (d) Changes in the percentage of sickled cells in isolated SCD RBCs following incubation under different hypoxic conditions in the absence or presence of PEG-ADA, MRS1754, H-89 or GA. Data in panels (a-d) are presented as the mean±SEM where *p<0.05 relative to control samples and ** p<0.05 relative to untreated hypoxia samples. n=4-6. (e) Working model of excess adenosine signaling in sickling and novel mechanism-based therapies in SCD. Under hypoxic conditions, increased adenosine-mediated 2, 3-DPG production via A_{2B}R is detrimental by decreasing Hb O₂ binding affinity of HbS, resulting in more deoxy-HbS and more sickling, hemolysis and multiple tissue damage and dysfunction. Without interference, hemolysis and multiple tissue injury lead to increased release of ATP which is converted to adenosine by the combined action of the ecto-nucleotidases, CD39 and CD73. The use of PEG-ADA to lower adenosine levels or an A_{2B}R antagonist block receptor activation will reduce the production of erythrocyte 2,3-DPG and reduce sickling Strategies to reduce erythrocyte 2,3-DPG content represent potentially novel mechanism-based therapies for the treatment of SCD.

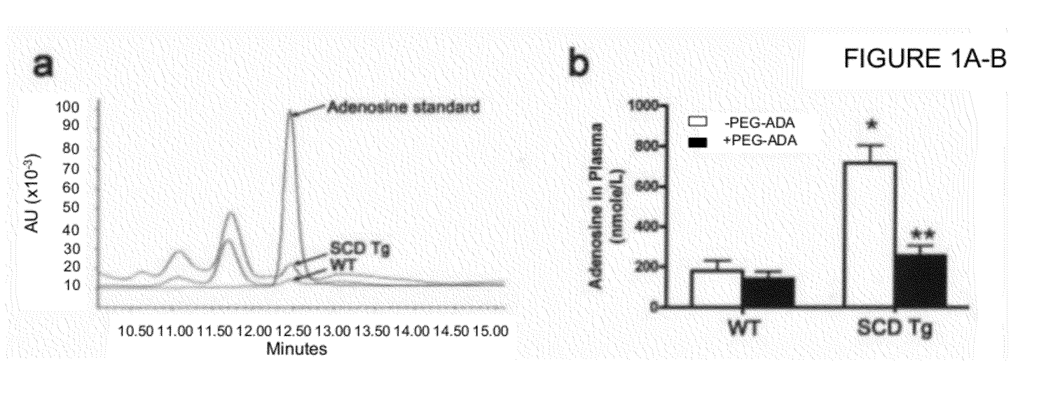

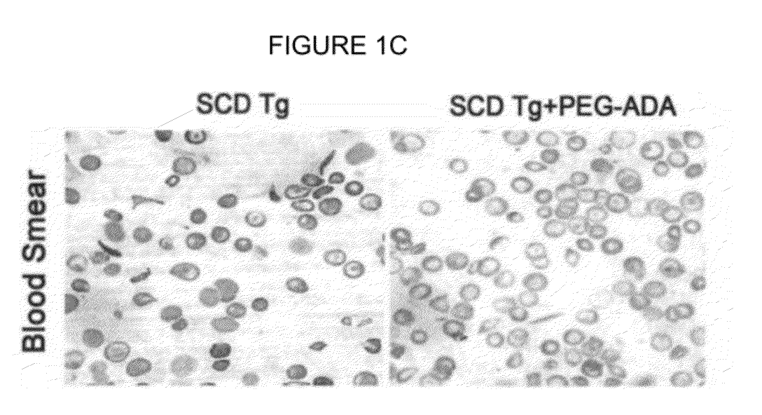

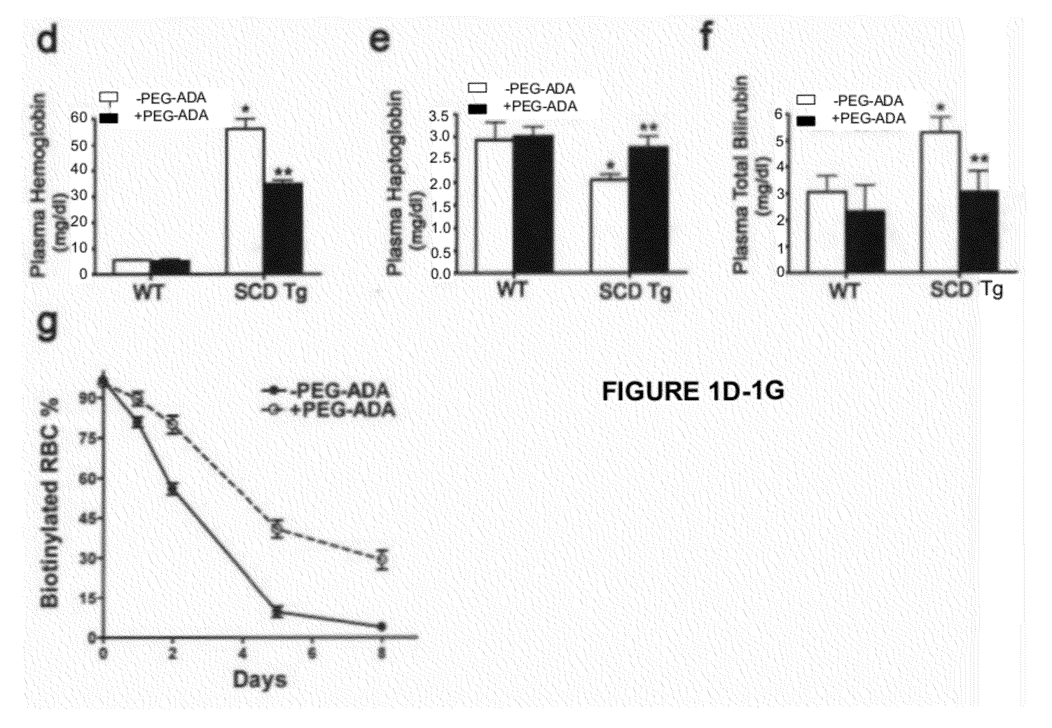

Supplementary FIG. 1. Metabolomic profiling of whole blood of wild type (WT) and SCD Tg mice. (a) Heat map showing the alteration of metabolites of 8 groups (including amino acids, carbohydrates, cofactors, TCA cycle, lipids, nucleosides & metabolites, peptides and xenobiotics). Shades of yellow and blue represent an increase and decrease of metabolite, respectively; relative to the median metabolite levels (see color scale). (b) Adenosine was highly increased in the whole blood of SCD Tg mice. (c) 2,3-DPG, an erythrocyte specific metabolite was also increased in the whole blood of SCD Tg mice. P<0.05, versus WT mice. n=6 for each group.

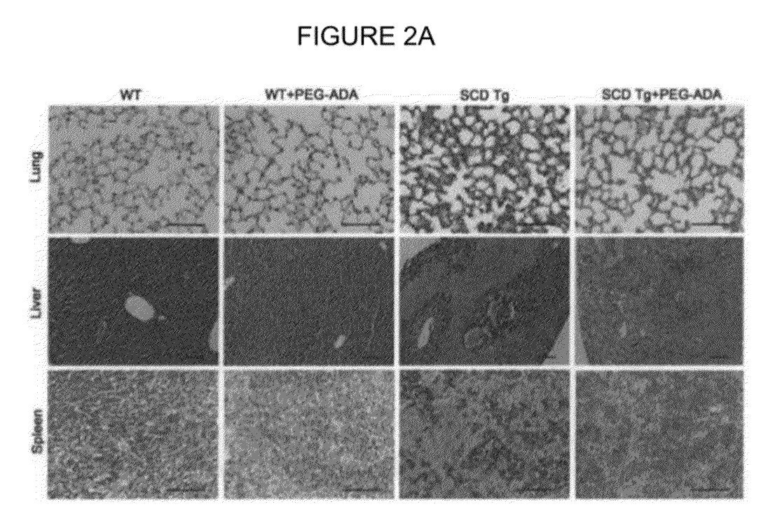

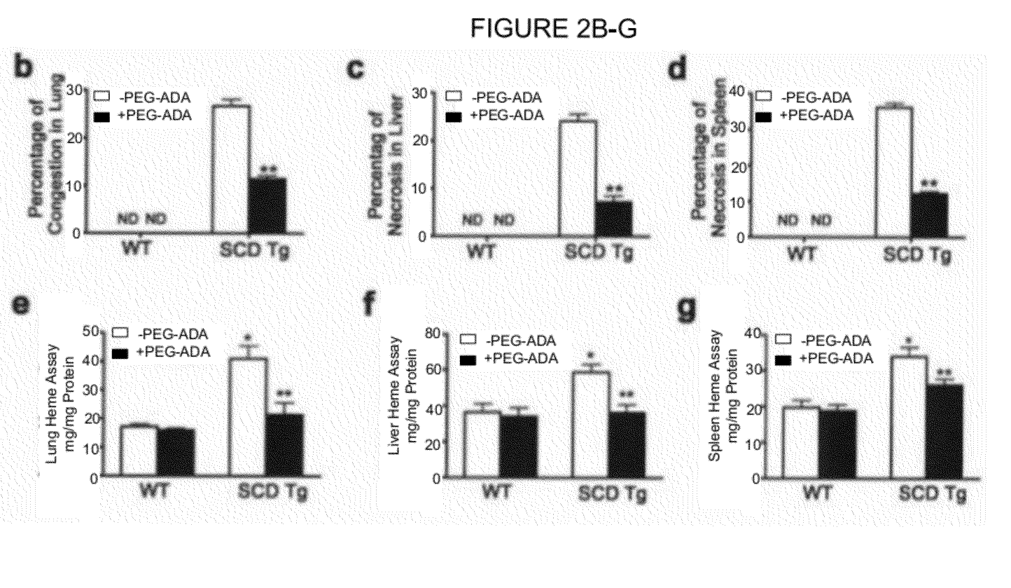

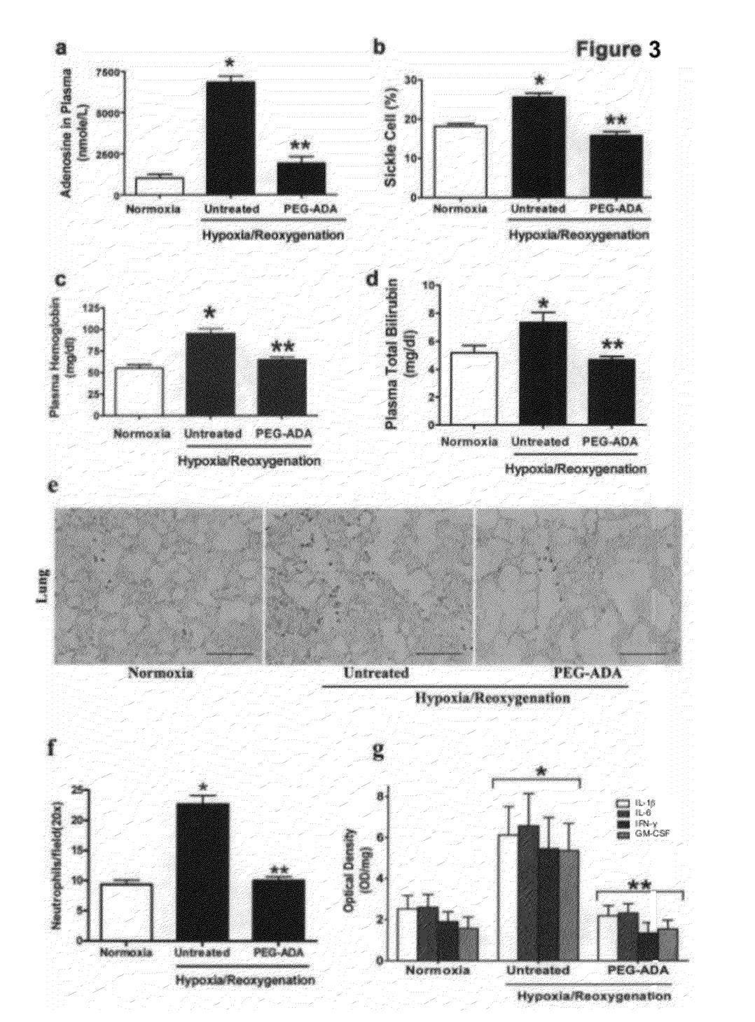

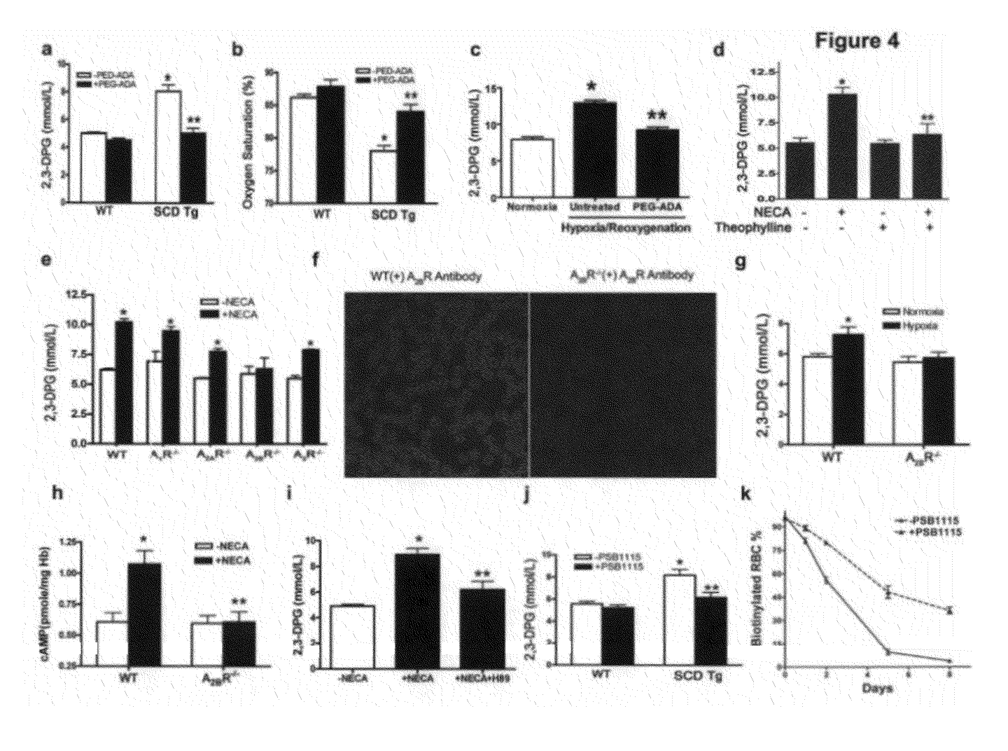

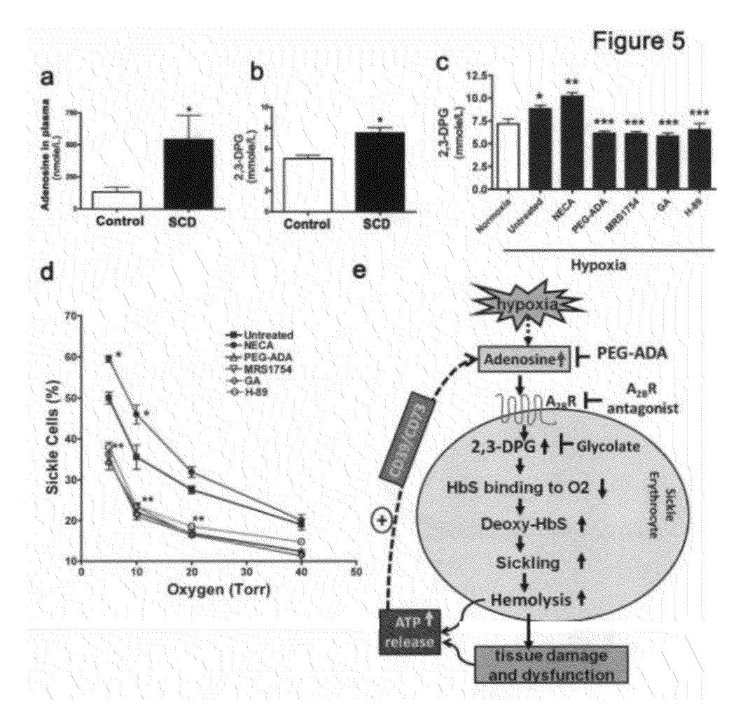

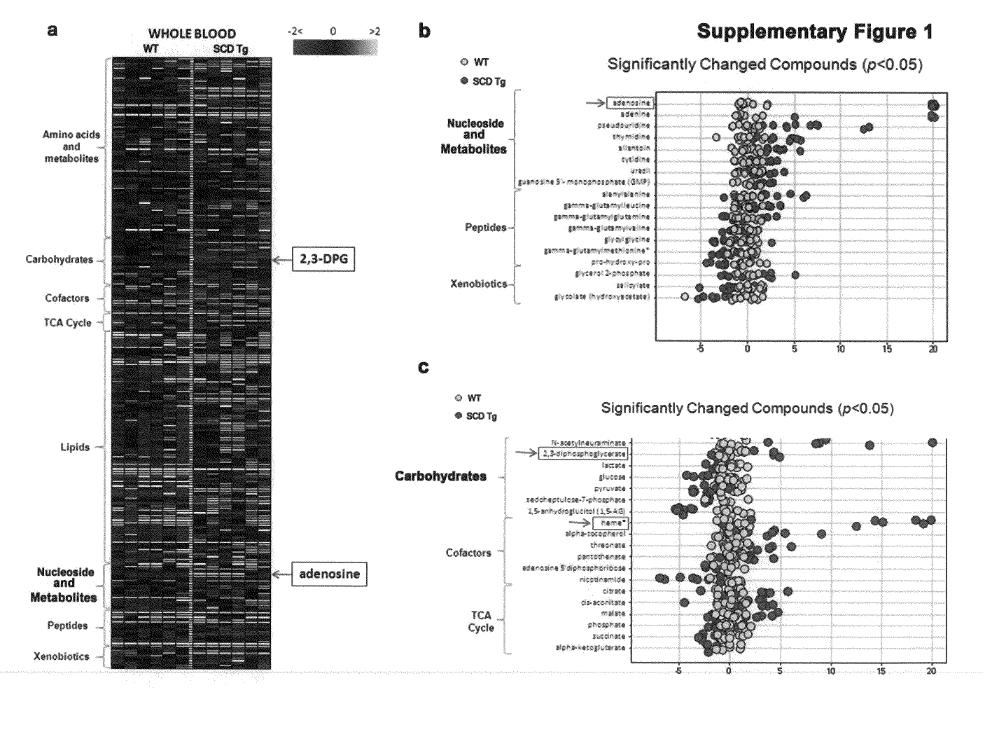

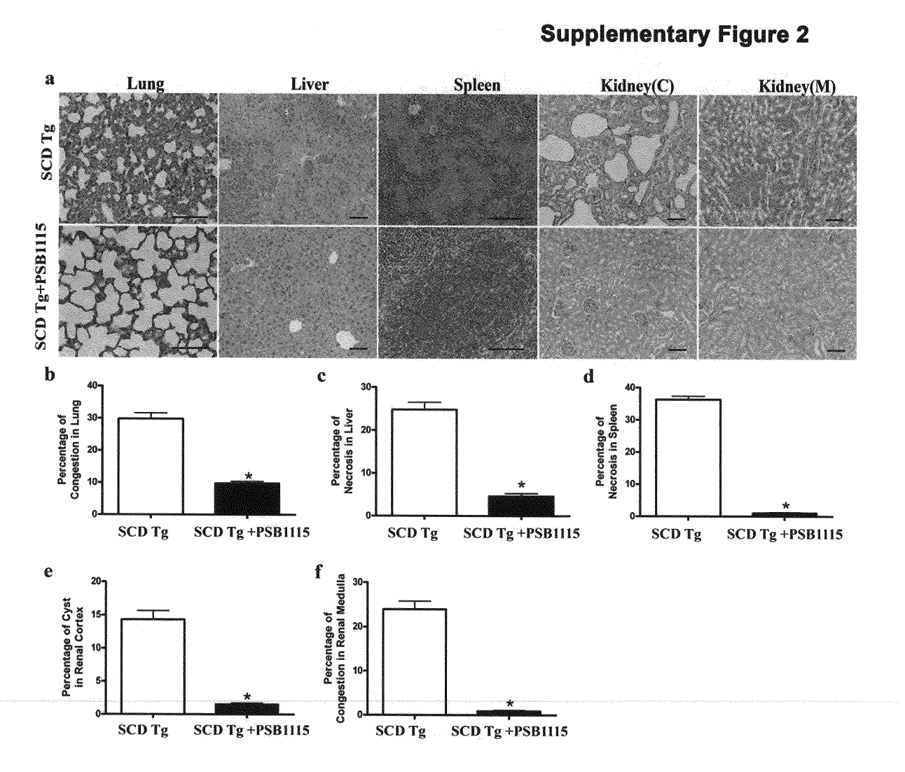

Supplementary FIG. 2. In vivo effects of PSB1115 chronic treatment in multiple organ damage associated with SCD Tg mice. (a) Histological analysis of lung, liver, spleen and kidneys of SCD Tg mice treated with or without PSB1115. Tissues were obtained from SCD Tg mice following 8 weeks with or without PSB1115 treatment. The results show significant congestion, vascular damage and necrosis in the lungs, livers and spleens of SCD Tg mice that was reduced by PSB1115 treatment. In addition, the microinfarction and cysts seen in renal cortex (C) and congestion in renal medulla (M) of SCD Tg mice were significantly decreased by 8-week PSB1115 chronic treatment. (b-f) Semiquantitative analysis of histological changes generated using Image-Plus Pro software in multiple tissues of the mice.

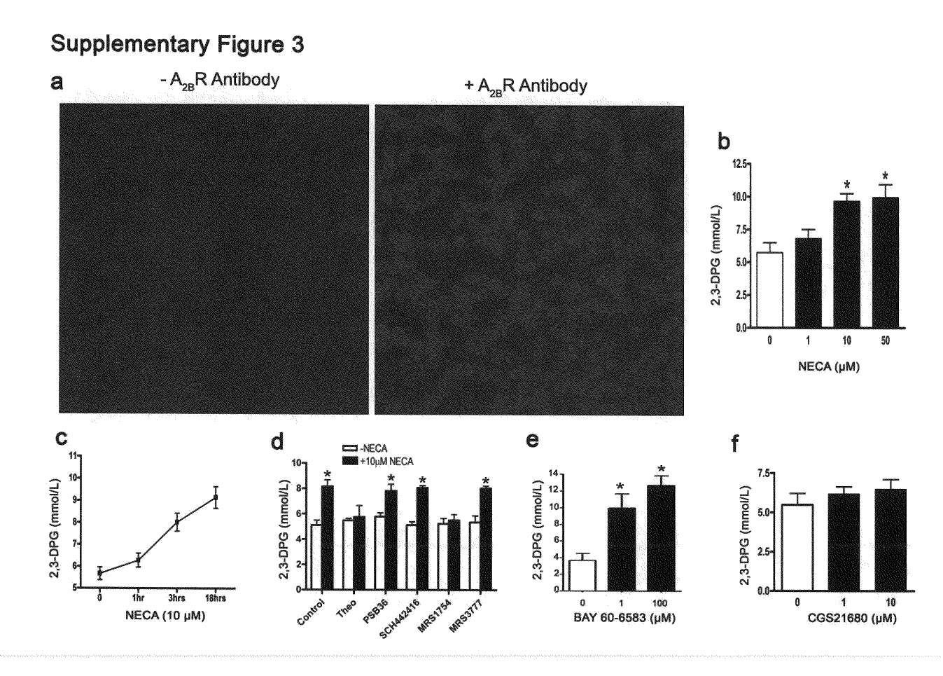

Supplementary FIG. 3. Adenosine-mediated increased 2,3-DPG levels requires A_{2B}R in normal human RBCs. (a) Immunostaining of A_{2B}R on surface of normal human RBCs. (b) Dose-dependent induction of 2,3-DPG by NECA in normal human RBCs. (c) Time-dependent induction of 2,3-DPG by NECA in normal human RBCs. (d) NECA-mediated induction of 2,3-DPG requires adenosine A2BR signaling. (e) Stimulation of 2,3-DPG production in human RBCs by A_{2B}R agonist, BAY 60-6583. (f) Lack of stimulation of 2,3-DPG production in human RBCs by A_{2A}R agonist, CGS21680. Data are presented as the mean±SEM where *p<0.05 relative to control samples untreated samples. n=4-6.

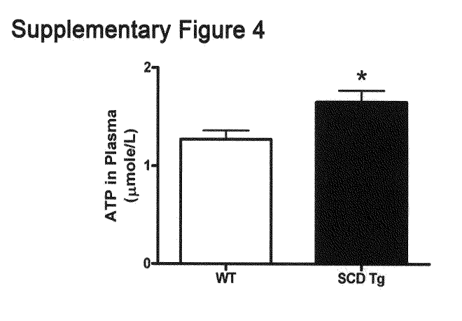

Supplementary FIG. 4. ATP level in plasma of wild type (WT) and SCD Tg mice. ATP levels in the plasma of WT and SCD Tg mice were analyzed. Results indicate that ATP concentrations in plasma from SCD Tg mice were significantly increased compared with that from WT mice. Data are presented as the mean±SEM where *p<0.05, versus WT mice. n=4-6.

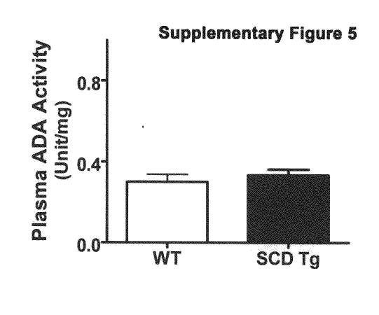

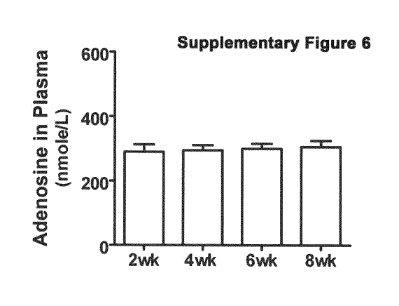

Supplementary FIG. 5. ADA activity in the plasma of wild type (WT) and SCD Tg mice. ADA activity in the plasma of WT and SCD Tg mice was analyzed. Results indicate that ADA activity in the plasma of SCD Tg was not significantly different from that of WT mice. Data are presented as the mean±SEM. n=5.

Supplementary FIG. 6. Adenosine levels in the plasma of SCD Tg mice with PEG-ADA treatment at different time points. SCD Tg mice were treated with PEG-ADA for 8 weeks at 2.5 U/week. Adenosine levels were measured at different time points. The results indicate that weekly injection of PEG-ADA lowered adenosine to a steady level by two weeks. N=6 for each time point.

### DETAILED DESCRIPTION

It is to be understood that both the foregoing general description and the following detailed description are exemplary and explanatory only, and are not restrictive of the invention, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that comprise more than one unit unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

One or more specific embodiments of the present invention are described below. The present invention is not limited to the embodiments and illustrations contained herein, but include modified forms of those embodiments including portions of the embodiments and combinations of elements of different embodiments as come within the scope of the appended claims. In the development of any such actual implementation, as in any engineering or design project, numerous implementation-specific decisions must be made to achieve the developers' specific goals, such as compliance with system-related and business related constraints, which may vary from one implementation to another. Moreover, such a development effort might be complex and time consuming, but would nevertheless be a routine undertaking of design, fabrication, and manufacture for those of ordinary skill having the benefit of this disclosure. Nothing in this application is considered critical or essential to the present invention unless explicitly indicated as being “critical” or “essential.”

As used herein, and unless otherwise indicated, the terms “treat”, “treating”, and “treatment” contemplate an action that occurs while a patient is suffering from SCD that reduces or reverses the severity of one or more symptoms or effects of SCD or an associated disease or disorder. Where the context allows, the terms “treat”, “treating”, and “treatment” also refers to actions taken toward ensuring that individuals at increased risk of SCD or associated symptoms are able to receive appropriate neurosurgical or other medical intervention prior to onset of SCD. As used herein, and unless otherwise indicated, the terms “prevent”, “preventing”, and “prevention” contemplate an action that occurs before a patient begins to suffer from SCD which t delays the onset of, and/or inhibits or reduces the severity of, SCD. As used herein, and unless otherwise indicated, the terms “manage”, “managing”, and “management” encompass preventing, delaying, or reducing the severity of a recurrence of SCD in a patient who has already suffered from such a disease or condition. The terms encompass modulating the threshold, development, and/or duration of the SCD or changing how a patient responds to the SCD.

As used herein, and unless otherwise specified, a “therapeutically effective amount” of a compound or composition is an amount sufficient to provide any therapeutic benefit in the treatment or management of SCD or to delay, reverse or minimize one or more symptoms associated with SCD. A therapeutically effective amount of a compound or composition means an amount of the compound or composition, alone or in combination with one or more other therapies and/or therapeutic agents that provides any therapeutic benefit in the treatment or management of SCD, or related and associated diseases or disorders. The term “therapeutically effective amount” can encompass an amount that alleviates SCD, improves or reduces RBC sickling and SCD or, improves overall therapy, or enhances the therapeutic efficacy of another therapeutic agent.

As used herein, and unless otherwise specified, a “prophylactically effective amount” of a compound or composition is an amount sufficient to prevent, reverse or delay the onset of RBC sickling and SCD, or one or more symptoms associated with SCD, or prevent or delay its recurrence. A prophylactically effective amount of a compound or composition means an amount of the compound or composition, alone or in combination with one or more other treatment and/or prophylactic agent that provides a prophylactic benefit in the prevention of SCD or associated disorders. The term “prophylactically effective amount” can encompass an amount that prevents SCD, improves overall prophylaxis, or enhances the prophylactic efficacy of another prophylactic agent.

In accordance with certain embodiments, methods and compositions are provided for preventing, treating or reducing the severity of SCD or a SCD associated symptom in a mammal, such as a human, having or prone to having SCD or a SCD associated symptoms. SCD associated symptoms include, but are not limited to, erythrocyte (RBC) sickling, oxygen release, increased HbS polymerization, hemolysis, tissue congestion and organ damage or dysfunction.

In certain embodiments the methods comprise administering to a host mammal a therapeutically effective amount of a composition that reduces adenosine signaling, alone or in combination. In some embodiments, the composition that reduces adenosine signaling does so by reducing the levels of adenosine. Reduction in the levels of adenosine can occur as a result of increasing the degradation of adenosine. In some embodiments, a composition that increases the degradation of adenosine comprises adenosine deaminase, bovine adenosine deaminase or human adenosine deaminase. Compositions of adenosine deaminase can comprise naturally occurring isolated and purified native protein or recombinant protein produced using molecular biologic techniques. In additional embodiments the adenosine deaminase is PEGylated. In other embodiments PEGylated adenosine deaminase is used in the manufacture of a medicament for the treatment of SCD or SCD associated symptoms. In some embodiments, a method is provided to selectively inhibit adenosine signaling in a human host with SCD or SCD associated symptoms, said method comprising administering a composition that does not result in significant toxic side effects and decreases adenosine signaling by selectively reducing the activity of adenosine in a human host in need of such a treatment.

In additional embodiments compositions that reduce adenosine signaling can do so by decreasing adenosine production, some such compositions are 5′-nucleotidase inhibitors, include but are not limited to ARL67156, APOPCP and others that can be delivered by injection. In alternative embodiments, adenosine signaling can be reduced by compositions that reduce adenosine receptor activity. In some embodiments, adenosine signaling can be reduced by inhibiting or blocking the adenosine receptor using antibodies, antibody fragments or aptamers. In other embodiments compositions that reduce adenosine signaling comprise adenosine receptor antagonists with broad activity such as, but not limited to, theophyline. In other embodiments, compositions that reduce adenosine signaling include those that act specifically through the A_{2B} adenosine receptor (A_{2B}R) or the A₂ₐ adenosine receptor. Such compositions that act on the A_{2B}R-receptor include, but are not limited to, MRS1706, CVT-6883 and PSB115. Compositions that act on the A2a receptor include, but is not limited to, ZM241385. Targeting this signaling pathway with PEG-ADA or other compositions that lower adenosine levels or A_{2B}R antagonists that decrease signaling and result in subsequent depletion of 2,3-DPG content provides a mechanism-based therapy for this devastating hemolytic disease.

A2B adenosine receptor antagonists are well known and have been described in, among other publications, U.S. Pat. Nos. 5,516,894, 5,599,671, 5,854,081, 6,060,481, 6,117,878, 6,387,913, 6,770,651, 6,825,349, 6,894,021, 6,916,804, 6,977,300, 7,105,665, 7,125,993, 7,205,403, 7,238,700, 7,304,070, 7,317,017, 7,335,655, 7,449,473, 7,470,697, 7,517,888, 7,521,554, 7,625,881, 7,691,825, 7,767,685, 7,795,268, 7,795,269, and 7855202; and U.S. Patent Publication Nos.: US20030064999, US20030139428, US20030207879, US20030229106, US20040209899, US20050038045, US20050119287, US20050101778, US20050261316, US20060058322, US20060142309, US20060281921, US20060293283, US20070059740, US20070219221, US20070265273, US20070281902, US20080045549, US20080085908, US20080153856, US20080176845, US20080194593, US20080275038, US20080318983, US20090023763, US20090082347, US20090137802, US20100222300, US20100056538, US20110130362, US20110160162, US20110184002 and US20110257127.

In still other embodiments, adenosine signaling can be reduced by inhibiting adenyl cyclase with compositions that include but is not limited to SQ22536. In still other embodiments, adenosine signaling can be reduced by inhibiting protein kinase A and such compositions include, but are not limited to, H-89 and PIK 14-22. In accordance with certain other embodiments a therapeutically effective amount of a composition that reduces adenosine signaling can be administered by injection via routes that include but are not limited to intravenous, intraperitoneal, intramuscular and intradermal. In other embodiments compositions can be administered orally or transdermally.

In one embodiment, a method of treating sickle cell disease, comprising administering to a person suffering from sickle cell disease a composition comprising an effective amount of at least one inhibitor of adenosine signaling and a pharmaceutically-acceptable carrier, wherein the inhibitor of adenosine signaling has at least one activity selected from the group consisting of decreasing adenosine levels in the mammal, inhibiting adenosine receptor activity in the mammal, and inhibiting signaling pathways downstream of an adenosine receptor in the mammal.

In some embodiments, the method described, wherein the at least one inhibitor of adenosine signaling is selected from the group consisting of adenosine deaminase (ADA), polyethylene-glycol modified adenosine deaminase (PEG-ADA), 5′-nucleotidase inhibitors, theophylline, adenosine receptor A_{2B} antagonists, adenylyl cyclase inhibitors, protein kinase A inhibitors, bisphosphoglycerate mutase inhibitors, glycolate, and salts and esters thereof.

In some embodiments, method described, wherein administering comprises one or more routes of administration selected from the group consisting of intravenous administration, intraperitoneal administration, intramuscular administration, intradermal administration, oral administration, and transdermal administration.

In another embodiment, a kit for treating sickle cell disease, comprising at least one inhibitor of adenosine signaling, a pharmaceutically-acceptable carrier, and instructions for the method described above.

In some embodiments, the kit described, wherein the at least one inhibitor of adenosine signaling is selected from the group consisting of adenosine deaminase (ADA), polyethylene-glycol modified adenosine deaminase (PEG-ADA), 5′-nucleotidase inhibitors, theophylline, adenosine receptor A_{2B} antagonists, adenylyl cyclase inhibitors, protein kinase A inhibitors, bisphosphoglycerate mutase inhibitors, glycolate, and salts and esters thereof.

In an additional embodiment, a method of manufacturing a medicament for the treatment of sickle cell disease, comprising combining an effective amount of at least one inhibitor of adenosine signaling and a pharmaceutically-acceptable carrier.

In some embodiments, the method described wherein the at least one inhibitor of adenosine signaling is selected from the group consisting of adenosine deaminase (ADA), polyethylene-glycol modified adenosine deaminase (PEG-ADA), 5′-nucleotidase inhibitors, theophylline, adenosine receptor A_{2B} antagonists, adenylyl cyclase inhibitors, protein kinase A inhibitors, bisphosphoglycerate mutase inhibitors, glycolate, and salts and esters thereof.

In some embodiments, the method described wherein, wherein the at least one inhibitor of adenosine signaling is an antagonist of the A_{2B} adenosine receptor.

In some embodiments, the method described wherein said antagonist of the A_{2B} adenosine receptor is drawn from the group consisting of theophylline, PSB36, SCH442416, MRS1754 and MRS37.

In some embodiments, methods of treating or preventing sickle cell disease symptoms in a mammal, comprising: administering to a person such symptoms a composition comprising an effective amount of at least one inhibitor of adenosine signaling and a pharmaceutically-acceptable carrier, wherein the inhibitor of adenosine signaling has at least one activity selected from the group consisting of decreasing adenosine levels in the mammal, inhibiting adenosine receptor activity in the mammal, and inhibiting signaling pathways downstream of an adenosine receptor in the mammal.

In some embodiments, the method described wherein, said symptoms are drawn from the group of sickling of erythrocytes, oxygen release, increased hemoglobin (HbS) polymerization, hemolysis, tissue congestion and organ damage.

In some embodiments, the method described wherein said symptom is the sickling of erythrocytes.

The above presents a simplified summary of the disclosed methods in order to provide a basic understanding of some aspects. This summary is not an exhaustive overview. It is not intended to identify key or critical elements or to delineate the scope of the invention. Its sole purpose is to present some concepts in a simplified form as a prelude to the more detailed description that is discussed later.

The inventors have discovered a new role for adenosine antagonists in the reversal of RBC sickling and related pathologies associated with SCD. Based on this information, they have further demonstrated and identified a previously unidentified role for compositions that reduce adenosine levels or antagonize the adenosine receptor to reduce adenosine signaling as treatments for SCD, a dangerous, painful and life-threatening hemolytic disease and associated disorders.

Methods and compositions for the treatment or prevention of SCD and associated disorders, disclosed herein, are based on these discoveries. The compositions comprise an effective amount of compounds or compositions whose application results in a reduction of adenosine singling. These compositions reduce adenosine induced signaling and thus prevent sickling of RBCs in response to hypoxia specifically by decreasing adenosine levels, either by increasing adenosine degradation, or decreasing adenosine production. Additionally, also described are compounds that act by antagonizing or blocking the A_{2B}R adenosine receptor, thus inhibiting adenosine mediated induction of 2,3-DPG in RBCs and treat or prevent RBC sickling and related pathologies associated with SCD and associated disorders.

Adenosine is a metabolic signaling molecule induced under energy depletion and ischemic/hypoxic conditions. In normal humans, increased adenosine under hypoxic conditions is believed to be beneficial due to its potent vasodilatory effect which increases blood flow to ischemic or hypoxic tissue. Surprisingly the inventors discovered that increased adenosine is, however, detrimental to individuals with SCD and that it promotes sickling of RBCs. These unexpected findings led to the discovery of novel underlying mechanism responsible for adenosine-mediated sickling of RBCs via A_{2B}R-mediated 2,3-DPG induction in erythrocytes. 2, 3-DPG is produced exclusively in RBCs to promote oxygen release from oxyhemoglobin (Sasaki, R. & Chiba, H. [Functions and metabolism of 2,3-bisphosphoglycerate in erythroid cells]. Tanpakushitsu kakusan koso 28, 957-973, 1983; Sasaki, R. & Chiba, H. Role and induction of 2,3-bisphosphoglycerate synthase. Molecular and Cellular Biochemistry 53-54, 247-256, 1983). Previous studies indicated that 2,3-DPG was elevated and involved in erythrocyte sickling under hypoxic conditions (Poillon, W. N., Kim, B. C., Labotka, R. J., Hicks, C. U. & Kark, J. A. Antisickling effects of 2,3-diphosphoglycerate depletion. Blood 85, 3289-3296, 1995; Poillon, W. N. & Kim, B. C. 2,3-Diphosphoglycerate and intracellular pH as interdependent determinants of the physiologic solubility of deoxyhemoglobin S. Blood 76, 1028-1036, 1990; Poillon, W. N., Kim, B. C., Welty, E. V. & Walder, J. A. The effect of 2,3-diphosphoglycerate on the solubility of deoxyhemoglobin S. Archives of Biochemistry and Biophysics 249, 301-305,1986). However, the molecular mechanism responsible for 2,3-DPG induction and its role in the pathogenesis in vivo of this molecule in SCD are previously unidentified. The presently disclosed findings in both human RBCs in vitro and mouse RBCs in vivo provide evidence that elevated adenosine induces 2,3-DPG production and that this elevated 2,3-DPG contributes to the sickling of RBCs by promoting oxygen release by RBCs. The present disclosure also provides genetic and pharmacological evidence that increased adenosine functions through A_{2B}R signaling to induce 2,3-DPG in normal RBCs from both humans and mice. However, in contrast to findings in normal humans, in humans with SCD it was determined that the A_{2B}R is the major receptor responsible for 2,3-DPG induction that and 2,3-DPG mediates RBC sickling induced by excess adenosine. The present disclosure therefore describes compelling evidence supporting the concept that adenosine is a normal physiological regulator for 2,3-DPG induction which promotes oxygen release in hypoxic tissues. However, in patients with SCD, this normally beneficial effect of increased oxygen release facilitates deoxy-HbS polymerization and subsequent RBC sickling.

Although SCD is the first genetic disorder for which a causative mutation was identified at the molecular level and one of the most prevalent autosomal recessive disorders worldwide, the medical community is unable to control HbS polymerization and erythrocyte sickling, which are central to the pathophysiology of the disease. By understanding the molecular events involved in the pathogenesis of erythrocyte sickling and HbS polymerization, the inventors were better positioned to develop novel therapeutic strategies to treat this disease.

It is well-know that hypoxic conditions will promote deoxygenation and subsequent polymerization of HbS which results in RBC sickling, resulting hemolysis, and eventually organ damage. Multiple factors and metabolites are altered in response to hypoxia and each may contribute to pathogenesis of the disease. To identify intracellular metabolites that may contribute to sickling and thereby exacerbate disease pathogenesis the inventors measured and compared a series of major metabolites in the whole blood of control individuals and SCD patients.

The present disclosure provides a discovery that provides new insight regarding the role and molecular mechanism of adenosine signaling in SCD, HbS polymerization, RBC sickling and hemolysis, and related pathologies and identifies compounds or compositions that inhibit adenosine signaling and prevent, reverse or treat SCD. Herein described for the first time is the finding that both humans and mice with SCD have elevated levels of circulating adenosine and provided both in vitro human studies and in vivo mouse evidence that increased adenosine is detrimental by promoting RBC sickling. Thus, under hypoxic conditions elevated adenosine and sickling act in a malicious cycle, eventually leading to multiple organ damage and dysfunction in SCD. The present disclosure describes methods and compositions that can be used to disrupt adenosine signaling either by lowing adenosine concentrations, blocking the adenosine receptor or using adenosine receptor antagonists to interrupt this detrimental cycle.

Adenosine induced signaling, HbS polymerization and RBC sickling can be prevented by compounds and compositions that decrease adenosine levels by increasing adenosine degradation or decreasing adenosine production. Alternatively, compounds or compositions can antagonize the adenosine receptor and thus can be used to prevent adenosine induced signaling.

Adenosine-signaling and adenosine induced RBC sickling can be prevented by compounds that decrease adenosine levels by increasing adenosine degradation. One such composition is a PEGylated version of the adenosine deaminase (ADA: EC 3.5.4.4) enzyme. In 1990, the FDA approved the marketing of ADAGEN® (pegademase bovine) Injection, designated as an Orphan Drug, for the indication of enzyme replacement therapy for ADA deficiency in patients with severe combined immunodeficiency disease (SCID) who are not suitable candidates for, or who have failed bone marrow transplantation. ADAGEN® represents the first successful application of enzyme replacement therapy for an inherited disease and is a life-saving treatment option that provides predictable restoration of ADA activity to ADA SCID patients. The chemical name for ADAGEN® (pegademase bovine) Injection is (monomethoxypolyethylene glycol succinimidyl) 11-17-adenosine deaminase. It is a conjugate of numerous strands of monomethoxypolyethylene glycol (PEG), molecular weight 5,000, covalently attached to the enzyme adenosine deaminase (ADA). The ADA used in the manufacture of ADAGEN® (pegademase bovine) Injection is derived from bovine intestine.

Such a PEGylated version of adenosine deaminase or a PEGylated version of human adenosine deaminase, which will be more likely to avoid the potential adverse reaction of immunity that can develop when non human proteins are used repeatedly to treat human patients. These proteins can be isolated and purified native proteins as with ADAGEN® (pegademase bovine) Injection which is derived from bovine intestine, or can be isolated and purified recombinant proteins developed using molecular biological techniques (Ausubel et al., eds., 1989, Current Protocols in Molecular Biology, Vol. I, Green Publishing Associates, Inc., and John Wiley & Sons, Inc., N.Y). PEG-ADA enzyme therapy or A_{2B}R antagonism offers an opportunity to halt the progression of SCD and decrease the morbidity and mortality associated with this hemolytic disorder.

A variety of host-expression vector systems can be used to express the native and variant nucleotide sequences. Such expression systems also encompass engineered host cells that express a protein, or functional equivalent, in situ. Purification or enrichment of a protein from such expression systems can be accomplished using appropriate detergents and lipid micelles and methods well-known to those skilled in the art. However, such engineered host cells themselves may be used in situations where it is important not only to retain the structural and functional characteristics of a protein, but to assess biological activity, e.g., in certain drug screening assays.

The expression systems that may be used include, but are not limited to, microorganisms such as bacteria (e.g., E. coli, B. subtilis) transformed with recombinant bacteriophage DNA, plasmid DNA or cosmid DNA expression vectors containing nucleotide sequences; yeast (e.g., Saccharomyces, Pichia) transformed with recombinant yeast expression vectors containing nucleotide sequences; insect cell systems infected with recombinant virus expression vectors (e.g., baculovirus) containing nucleotide sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing nucleotide sequences; or mammalian cell systems (e.g., COS, CHO, BHK, 293, 3T3) harboring recombinant expression constructs containing nucleotide sequences and promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the protein product being expressed. For example, when a large quantity of such a protein is to be produced for the generation of pharmaceutical compositions of, or containing, a protein, or for raising antibodies to a protein, vectors that direct the expression of high levels of fusion protein products that are readily purified may be desirable. Such vectors include, but are not limited to, the E. coli expression vector pUR278 (Ruther et al., 1983, EMBO J. 2:1791), in which a protein coding sequence may be ligated individually into the vector in-frame with the lacZ coding region so that a fusion protein is produced; pIN vectors (Inouye and Inouye, 1985, Nucleic Acids Res. 13:3101-3109; Van Heeke and Schuster, 1989, J. Biol. Chem. 264:5503-5509); and the like. pGEX® vectors (Pharmacia® or American Type Culture Collection®) can also be used to express foreign polypeptides as fusion proteins with glutathione S-transferase (GST). In general, such fusion proteins are soluble and can easily be purified from lysed cells by adsorption to glutathione-agarose beads, followed by elution in the presence of free glutathione. The PGEX® vectors are designed to include thrombin or factor Xa protease cleavage sites so that the cloned target expression product can be released from the GST moiety.

In an exemplary insect system, Autographa californica nuclear polyhedrosis virus (AcNPV) is used as a vector to express foreign polynucleotide sequences. The virus grows in Spodoptera frugiperda cells. A protein coding sequence can be cloned individually into a non-essential region (for example the polyhedrin gene) of the virus and placed under control of an AcNPV promoter (for example the polyhedrin promoter). Successful insertion of a protein coding sequence will result in inactivation of the polyhedrin gene and production of non-occluded recombinant virus (i.e., virus lacking the proteinaceous coat coded for by the polyhedrin gene). These recombinant viruses are then used to infect Spodoptera frugiperda cells in which the inserted sequence is expressed (e.g., see Smith et al., 1983, J. Virol. 46:584; and U.S. Pat. No. 4,215,051).

In mammalian host cells, a number of viral-based expression systems may be utilized. In cases where an adenovirus is used as an expression vector, the nucleotide sequence of interest may be ligated to an adenovirus transcription/translation control complex, e.g., the late promoter and tripartite leader sequence. This chimeric sequence may then be inserted in the adenovirus genome by in vitro or in vivo recombination. Insertion in a non-essential region of the viral genome (e.g., region E1 or E3) will result in a recombinant virus that is viable and capable of expressing a protein product in infected hosts (e.g., see Logan and Shenk, 1984, Proc. Natl. Acad. Sci. USA 81:3655-3659). Specific initiation signals may also be required for efficient translation of inserted nucleotide sequences. These signals include the ATG initiation codon and adjacent sequences. In cases where an entire protein gene or cDNA, including its own initiation codon and adjacent sequences, is inserted into the appropriate expression vector, no additional translational control signals may be needed. However, in cases where only a portion of a protein coding sequence is inserted, exogenous translational control signals, including, perhaps, the ATG initiation codon, may be provided. Furthermore, the initiation codon should be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bitter et al., 1987, Methods in Enzymol. 153:516-544).

In addition, a host cell strain may be chosen that modulates the expression of the inserted sequences, or modifies and processes the expression product in the specific fashion desired. Such modifications (e.g., glycosylation) and processing (e.g., cleavage) of protein products may be important for the function of the protein. Different host cells have characteristic and specific mechanisms for the post-translational processing and modification of proteins and expression products. Appropriate cell lines or host systems can be chosen to ensure the desired modification and processing of the foreign protein expressed. To this end, eukaryotic host cells that possess the cellular machinery for the desired processing of the primary transcript, glycosylation, and phosphorylation of the expression product may be used. Such mammalian host cells include, but are not limited to, CHO, VERO, BHK, HeLa, COS, MDCK, 293, 3T3, W138, and in particular, human cell lines.

For long-term, high-yield production of recombinant proteins, stable expression is preferred. For example, cell lines that stably express the protein sequences described herein are engineered. Rather than using expression vectors that contain viral origins of replication, host cells can be transformed with DNA controlled by appropriate expression control elements (e.g., promoter, enhancer sequences, transcription terminators, polyadenylation sites, etc.), and a selectable marker. Following the introduction of the foreign DNA, engineered cells may be allowed to grow for 1-2 days in an enriched media, and then switched to a selective media. The selectable marker in the recombinant plasmid confers resistance to the selection and allows cells to stably integrate the plasmid into their chromosomes and grow to form foci, which in turn can be cloned and expanded into cell lines. This method may advantageously be used to engineer cell lines that express a protein product. Such engineered cell lines may be particularly useful in screening and evaluation of compounds that affect the endogenous activity of a protein product.

A number of selection systems may be used, including, but not limited to, the Herpes simplex virus thymidine kinase (Wigler et al., 1977, Cell 11:223), hypoxanthine-guanine phosphoribosyltransferase (Szybalska and Szybalski, 1962, Proc. Natl. Acad. Sci. USA 48:2026), and adenine phosphoribosyltransferase (Lowy et al., 1980, Cell 22:817) genes, which can be employed in tk⁻, hgprt⁻ or aprt⁻ cells, respectively. Also, antimetabolite resistance can be used as the basis of selection for the following genes: dhfr, which confers resistance to methotrexate (Wigler et al., 1980, Proc. Natl. Acad. Sci. USA 77:3567; O'Hare et al., 1981, Proc. Natl. Acad. Sci. USA 78:1527); gpt, which confers resistance to mycophenolic acid (Mulligan and Berg, 1981, Proc. Natl. Acad. Sci. USA 78:2072); neo, which confers resistance to the aminoglycoside G-418 (Colberre-Garapin et al., 1981, J. Mol. Biol. 150:1); and hygro, which confers resistance to hygromycin (Santerre et al., 1984, Gene 30:147).

Alternatively, any fusion protein can be readily purified by utilizing an antibody specific for the fusion protein being expressed. Another exemplary system allows for the ready purification of non-denatured fusion proteins expressed in human cell lines (Janknecht et al., 1991, Proc. Natl. Acad. Sci. USA 88:8972-8976). In this system, the sequence of interest is subcloned into a vaccinia recombination plasmid such that the sequence's open reading frame is translationally fused to an amino-terminal tag consisting of six histidine residues. Extracts from cells infected with recombinant vaccinia virus are loaded onto Ni²⁺-nitriloacetic acid-agarose columns, and histidine-tagged proteins are selectively eluted with imidazole-containing buffers.

Alternatively adenosine signaling can be reduced by using compositions that reduce adenosine receptor activity. Such compositions include, but are not limited to, those that inhibit or blocking the adenosine receptor and thus its activity and include, but are not limited to antibodies, antibody fragments and aptamers. Also encompassed are antibodies and anti-idiotypic antibodies (including Fab fragments) that bind and or block adenosine receptors and inhibit its activity, antagonists and agonists of the adenosine receptor activity, as well as compounds or nucleotide constructs that inhibit adenosine receptor activity (transcription factor inhibitors, antisense and ribozyme molecules, or open reading frame sequence or regulatory sequence replacement constructs) or induction of 2,3-DPG.

The use of antibodies that selectively bind to one or more epitopes of adenosine receptora or epitopes of conserved variants of adenosine receptors or to splice variant isoforms of adenosine receptor and their fragments are also contemplated, particularly for use in the immunoassays described herein. Antibodies for use in these immunoassays include those available commercially. Such antibodies include, but are not limited to, polyclonal antibodies, monoclonal antibodies (mAbs), humanized antibodies, human-engineered antibodies, fully human antibodies, chimeric antibodies, single chain antibodies, Fab fragments, F(ab′)2 fragments, fragments produced by a Fab expression library, anti-idiotypic (anti-Id) antibodies, catalytic antibodies, and epitope-binding fragments of any of the above. In some applications, the antibodies, or fragments thereof, will preferentially bind to native or variant adenosine receptor, as opposed to other proteins. In such cases, the antibodies, or fragments thereof, selectively bind to native, recombinant or variant adenosine receptor with a higher affinity or avidity than they bind to other proteins (“Antibodies: A Laboratory Manual” (Harlow and Lane, eds., Cold Spring Harbor Laboratory Press, Cold Spring Harbor, N.Y., 1988).

Adenosine receptor protein products can also be used as therapeutics. For example, soluble derivatives such as adenosine receptor protein peptides/domains corresponding to variant adenosine receptor proteins, fusion proteins products (especially adenosine receptor protein-Ig fusion proteins, i.e., fusions of a adenosine receptor protein, or a domain of a adenosine receptor protein protein, to an IgFc), adenosine receptor protein antibodies and anti-idiotypic antibodies (including Fab fragments), antagonists or agonists (including compounds that modulate or act on downstream targets in a adenosine receptor-mediated pathway) can be used to directly treat SCD and associated symptoms or disorders. For instance, the administration of an effective amount of a soluble adenosine receptor protein, a adenosine receptor protein-IgFc fusion protein, or an anti-idiotypic antibody (or its Fab) that mimics or block adenosine receptor activity and effectively antagonize an endogenous adenosine receptor activity. Nucleotide constructs encoding such adenosine receptor protein products can be used to genetically engineer host cells to express such products in vivo; these genetically engineered cells function as “bioreactors” in the body delivering a continuous supply of adenosine receptor protein, adenosine receptor peptide, or adenosine receptor fusion protein to the body. Nucleotide constructs encoding functional adenosine receptor proteins, mutant adenosine receptor proteins, as well as antisense and ribozyme molecules, can also be used in “gene therapy” approaches for the modulation of adenosine receptor activity. Thus, also encompassed are pharmaceutical formulations and methods for treating SCD related disorders.

Also contemplated are isolated or purified proteins or polypeptides. The phrase “substantially isolated” or “substantially pure” protein or polypeptide is meant to describe a protein or polypeptide that has been separated from at least some of those components that naturally accompany it. Typically, the protein or polypeptide is substantially isolated or pure when it is at least 60%, by weight, free from the proteins and other naturally-occurring organic molecules with which it is naturally associated in vivo. Preferably, the purity of the preparation is at least 75%, more preferably at least 90%, and most preferably at least 99%, by weight. A substantially isolated or pure protein or polypeptide may be obtained, for example, by extraction from a natural source, by expression of a recombinant nucleic acid encoding the protein or polypeptide, or by chemically synthesizing the protein or polypeptide.

Purity can be measured by any appropriate method, e.g., column chromatography such as immunoaffinity chromatography using an antibody specific for the protein or polypeptide, polyacrylamide gel electrophoresis, or HPLC analysis. A protein or polypeptide is substantially free of naturally associated components when it is separated from at least some of those contaminants that accompany it in its natural state. Thus, a polypeptide that is chemically synthesized or produced in a cellular system different from the cell from which it naturally originates will be, by definition, substantially free from its naturally associated components. Accordingly, substantially isolated or pure proteins or polypeptides include eukaryotic proteins synthesized in E. coli, other prokaryotes, or any other organism in which they do not naturally occur.

### Pharmaceutical Preparations and Methods of Administration

Compositions that are determined to affect adenosine signaling or adenosine activity can be administered to a patient at therapeutically effective doses to treat or ameliorate medical conditions and symptoms associated with SCD, for example, including, but are not limited to, oxygen release, increased HbS polymerization, RBC sickling, hemolysis, tissue congestion and disruption and organ damage or failure. A therapeutically effective dose refers to that amount of the compound sufficient to result in any amelioration or retardation of disease symptoms, or development and cell differentiation or proliferation disorders.

### Effective Dose

Generally, the effective dose of a compound can be determined as a matter of routine experimentation by the person of ordinary skill in the art, in view of the present disclosure and particularly the following discussion. As should be apparent, the effective dose of a compound will depend on the identity of the compound, among other factors. Toxicity and therapeutic efficacy of such compositions can be determined by standard pharmaceutical procedures in cell cultures or experimental animals, e.g., for determining the TD₅₀ (the dose producing behavioral toxicity to 50% of the population) and the ED₅₀ (the dose therapeutically effective in 50% of the population). The dose ratio between toxic and therapeutic effects is the therapeutic index and it can be expressed as the ratio TD₅₀/ED₅₀. Compositions which exhibit large therapeutic indices, such as humanized adenosine antibodies, or PEGylated human ADA are preferred. While compositions that exhibit toxic side effects can be used, care should be taken to design a delivery system that targets such compositions to the site of affected tissue in order to minimize potential damage to uninfected cells and, thereby, reduce side effects.

The data obtained from cell culture assays and animal studies can be used in formulating a range of dosage for use in humans when dealing with non-approved therapies. The dosage of such compositions lies preferably within a range of circulating concentrations that include the ED₅₀ with little or no toxicity. The dosage may vary within this range depending upon the dosage form employed and the route of administration utilized. For any compositions used in the disclosed method, the therapeutically effective dose can be estimated initially from cell culture assays. A dose may be formulated in animal models to achieve a circulating plasma concentration range that includes the IC₅₀ (i.e., the concentration of the test compositions which achieves a half-maximal inhibition of symptoms) as determined in cell culture. Such information can be used to more accurately determine useful doses in humans. Levels in plasma may be measured, for example, by high performance liquid chromatography.

When the therapeutic treatment of disease is contemplated, the appropriate dosage may also be determined using animal studies to determine the maximal tolerable dose, or MTD, of a bioactive agent per kilogram weight of the test subject. In general, at least one animal species tested is mammalian. Those skilled in the art regularly extrapolate doses for efficacy and avoiding toxicity to other species, including human. Before human studies of efficacy are undertaken, Phase I clinical studies in normal subjects help establish safe doses.

Additionally, the bioactive agent may be complexed with a variety of well-established compounds or compositions or structures that, for instance, enhance the stability of the bioactive agent, or otherwise enhance its pharmacological properties (e.g., increase in vivo half-life, reduce toxicity, etc.).

The above therapeutic agents will be administered by any number of methods known to those of ordinary skill in the art including, but not limited to, administration by inhalation; by subcutaneous (sub-q), intravenous (I.V.), intraperitoneal (I.P.), intramuscular (I.M.), intracranial, or intrathecal injection; or as a topically applied agent (transderm, ointments, creams, salves, eye drops, and the like).

### Formulations and Use

Pharmaceutical compositions for use in accordance with the presently disclosed methods can be formulated in a conventional manner using one or more physiologically acceptable carriers or excipients.

Thus, the compositions and their physiologically acceptable salts and solvates may be formulated for administration by inhalation or insufflation (either through the mouth or the nose) or oral, buccal, parenteral or rectal administration.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound.

For buccal administration the compositions may take the form of tablets or lozenges formulated in a conventional manner.

For administration by inhalation, the compounds for use according to the present methods are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compositions, and particularly humanized monoclonal antibodies, can be formulated for parenteral administration by injection, e.g., by bolus injection or continuous infusion. Formulations for injection can be presented in unit dosage form, e.g., in ampules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compositions can also be formulated as compositions for rectal administration such as suppositories or retention enemas, e.g., containing conventional suppository bases such as cocoa butter or other glycerides.

In addition to the formulations described previously, the compositions may also be formulated as a depot preparation. Such long acting formulations may be administered by implantation (for example, subcutaneously or intramuscularly) or by intramuscular injection. Thus, for example, the compositions may be formulated with suitable polymeric or hydrophobic materials (for example, as an emulsion in an acceptable oil) or ion exchange resins, or as sparingly soluble derivatives, for example, as a sparingly soluble salt. The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

In some embodiments, a kit comprising at least one inhibitor of adenosine signaling and a pharmaceutically-acceptable carrier, as described above. The kit further comprises instructions for using the at least one inhibitor of adenosine signaling to treat sickle cell disease. The instructions may be specific for one or more of the embodiments (such as formulations, routes of administration, etc.) described above. The kit may further comprise appropriate supplies for carrying out the instructions (e.g., a syringe and/or drip bag and associated tubing, and associated materials, suitable for administering a solution comprising the at least one inhibitor of adenosine signaling intravenously).

In some embodiments, the kit described, wherein the at least one inhibitor of adenosine signaling is selected from the group consisting of adenosine deaminase (ADA), polyethylene-glycol modified adenosine deaminase (PEG-ADA), 5′-nucleotidase inhibitors, theophylline, adenosine receptor A_{2B} antagonists, adenylyl cyclase inhibitors, protein kinase A inhibitors, bisphosphoglycerate mutase inhibitors, glycolate, and salts and esters thereof.

### The Use of Inhibitors of Adenosine Signaling to Treat Humans with Scd

In view of the foregoing in vivo evidence that indicates that inhibition of adenosine signaling, results in decreased SCD associated symptoms including, but are not limited to, RBC sickling, oxygen release, increased HbS polymerization, hemolysis, tissue congestion and disruption and organ damage or failure. While the in vivo data presented in the examples was obtained using SCD Tg mice, those of skill in the art will readily recognize that these observations will extend to other mammals including humans particularly in light of the in vitro confirming evidence presented using human normal and SCD patient primary cells. These findings further indicate that inhibitors (inter alia, antibodies, proteins, polypeptides, peptides or fragments thereof, genetic disruption by recombination, RNAi, aptamers, small molecule inhibitors or any other form of inhibitor known to the art) of the adenosine signaling pathway can reproduce the physiologic effects observed in mice and human tissue and demonstrate that inhibitors of the adenosine signaling pathway can be used to treat SCD and SCD associated symptoms. Using techniques described in this application and those known to the art, regarding pharmaceutical preparations and methods of administration it is clear that adenosine signaling pathway inhibitors, A_{2B}R antagonists, such as, but not limited to PEG-ADA, can be used in humans to treat SCD and SCD associated disorders.

Adenosine signaling inhibitors for treatment of SCD and associated disorders can be administered, said administration occurring daily, every other day, weekly, bi-weekly, monthly, bi-monthly, quarterly or once per year, by any suitable route of administration, including oral, subcutaneous and parenteral administration. Examples of parenteral administration include intravenous, intra-articular, intramuscular, intranasal, intraocular, inhaled and intraperitoneal.

Regardless of the manner of administration, the specific dose may be calculated according to such factors as body weight or body surface and based on finding in drug metabolism and pharmacokinetic (DMPK) analyses. Further refinement of the calculations necessary to determine the appropriate dosage for modulating adenosine signaling pathway mediated disorders, inter alia, SCD and SCD associated disorder, can readily be made by those of ordinary skill in the art without undue experimentation.

During the course of treatment, the effects of the adenosine signaling pathway inhibitors on SCD and SCD associated disorders can be monitored and evaluated using, for example, CBC and differentials to enumerated blood cells, sedimentation rates, cytokine levels and cell subpopulation analyses done on, peripheral blood or other sample, as appropriate based on symptoms, intuition or the results of other medical laboratory techniques available through most medical facilities and hospitals, such as CBC, FACS and clinical blood chemistry analysis and the assays and techniques which are present in the exemplary embodiments described in this disclosure.

In some embodiments, a method for preventing, treating or reducing the severity of sickle cell disease or a sickle cell disease associated symptom in a mammal having or prone to having sickle cell disease or a sickle cell disease associated symptom, the method comprising administering to a host mammal having or prone to having sickle cell disease or a sickle cell disease associated symptom, a therapeutically effective amount of a composition that reduces adenosine signaling, to prevent, treat or reduce RBC sickling and the severity of sickle cell disease or a sickle cell disease associated symptom.

In some embodiments, the method described, wherein said composition reduces adenosine signaling by reducing the levels of adenosine. In some embodiments, the method described, wherein said composition reduces the levels of adenosine by increasing degradation of adenosine. In some embodiments, the method described, wherein said composition comprises adenosine deaminase. In some embodiments, the method described, wherein said adenosine deaminase comprises purified native protein or recombinant adenosine deaminase protein. In some embodiments, the method described, wherein said adenosine deaminase comprises bovine adenosine deaminase. In some embodiments, the method described, wherein said adenosine deaminase comprises human adenosine deaminase. In some embodiments, the method described, wherein said adenosine deaminase is PEGylated. In some embodiments, the method described, wherein said composition reduces the levels of adenosine by decreasing adenosine production. In some embodiments, the method described, wherein said compositions comprise 5′-nucleotidase inhibitors. In some embodiments, the method described, wherein said 5′-nucleotidase inhibitors are selected from the group consisting of ARL67156 and APOPCP. In some embodiments, the method described, wherein said composition reduces adenosine signaling by reducing adenosine receptor activity. In some embodiments, the method described, wherein said composition comprises an adenosine receptor antagonist. In some embodiments, the method described, wherein said composition comprises theophylline. In some embodiments, the method described, wherein said adenosine receptor is receptor A_{2B}. In some embodiments, the method described, wherein said adenosine receptor is receptor Aₜₐ. In some embodiments, the method described, wherein said composition comprises an antagonist of adenosine receptor A_{2B}. In some embodiments, the method described, wherein said composition comprises an antagonist of adenosine receptor A_{2B} selected from the group consisting of MRS1706, CVT-6883 and PSB115. In some embodiments, the method described, wherein said composition comprises an antagonist of adenosine receptor A_{2B} selected from the group consisting of theophylline, PSB36, SCH442416, MRS1754 and MRS3777. In some embodiments, the method described, wherein said composition comprises ZM241385. In some embodiments, the method described, wherein said compositions that reduce adenosine signaling comprise adenyl cyclase inhibitors. In some embodiments, the method described, wherein said adenyl cyclase inhibitor is SQ22536. In some embodiments, the method described, wherein said compositions that reduce adenosine signaling comprise protein kinase A inhibitors. In some embodiments, the method described, wherein said protein kinase A inhibitor is H-89 or PIK₁₄₋₂₂. In some embodiments, the method described, wherein said composition is administered by injection by routes selected from the group consisting of intravenous, intraperitoneal, intramuscular, or intradermal. In some embodiments, the method described, wherein said composition is administered orally or transdermally. In some embodiments, the method described, wherein said composition is administered alone or in combination with another composition described above. In some embodiments, the method described wherein said composition inhibits or blocks the adenosine receptor. In some embodiments, the method described, wherein said composition is selected from the group consisting of antibodies, antibody fragments and aptamers. In some embodiments, the method described, wherein said mammal is a human. In some embodiments, the method described, wherein said sickle cell disease associated symptom is selected from the group consisting of erythrocyte sickling, oxygen release, increased HbS polymerization, hemolysis, tissue and organ damage or dysfunction. In some embodiments, a method for selectively inhibiting adenosine signaling in a human host with sickle cell disease or sickle cell disease associated symptoms, comprising administering a composition that decreases adenosine signaling by selectively reducing the activity of adenosine in a human host in need of such a treatment. In some embodiments, the method described, wherein said composition does not result in significant toxic side effects in the human host. In further embodiments, the methods described relate to the use of PEGylated adenosine deaminase in the manufacture of a medicament for the treatment of sickle cell disease or sickle cell disease associated symptoms.

The presently disclosed methods are not to be limited in scope by the specific embodiments described herein, which are intended as single illustrations of individual aspects of the methods, and functionally equivalent methods and components are within the scope of the invention. Indeed, various modifications of the described methods, in addition to those shown and described herein will become apparent to those skilled in the art from the foregoing description. Such modifications are intended to fall within the scope of the appended claims.

### EXAMPLES

### General Methodologies

Human Subjects Sickle cell disease patients in the steady state were identified by a hematologist on the faculty of the University of Texas Medical School at Houston. Patients participating in this study had no blood transfusion for at least 6 months before blood samples were collected. Half of the patients were treated with hydroxyurea. Control subjects were of African descent and were free of hematological diseases. Relevant clinical features of study patients are presented in Supplementary Table 2. The research protocol, which included informed consent from the subjects, was approved by the University of Texas Health Science Center at Houston Committee for the Protection of Human Subjects.

Blood Collection and Hematological Analysis. Approximately 1 ml of blood was withdrawn from a forearm vein of normal individuals and SCD patients. The blood was collected in 1.5 ml tube containing 10 μM dipyridamole, 10 μM α,β-methylene ADP and 10 μM 5′-deoxycoformycin (DCF), immediately dropped into liquid nitrogen and subsequently stored at −800 C for metabolic analysis. 4 ml of blood was collected with heparin as an anti-coagulant for 2,3-diphosphoglycerate (2,3-DPG) measurement. An additional 4 ml of blood was collected with EDTA as an anti-coagulant and used for morphological study, complete blood count (CBC) and hemoglobin electrophoresis and 1 ml of blood was aliquoted to 1.5 ml tubes containing 10 μM dipyridamole and 10 μM DCF for plasma adenosine assay.

Mice. Berkley sickle cell disease transgenic mice (SCD Tg mice), expressing exclusively human sickle hemoglobin (HbS) were purchased from The Jackson Laboratory Inc (Paszty, C. Transgenic and gene knock-out mouse models of sickle cell anemia and the thalassemias. Curr Opin Hematol 4, 88-93, 1997). C57BL/6 mice used as controls were purchased from Harlan Laboratories. Adenosine receptor deficient mice were obtained from the following sources: A₁R-deficient mice (Jurgen Schnermann, NIDDK, NIH); A_{2A}R-deficient mice (Jiang-Fan Chen, Boston University School of Medicine); A_{2B}R-deficient mice (Michael R. Blackburn, University of Texas-Houston Medical School); and A₃R-deficient mice (Marlene Jacobson, Merck Research Laboratories). All receptor deficient mice were backcrossed at least 10 generations onto the C57Blk/6J background and were genotyped according to established protocols (Sun, D., et al. Mediation of tubuloglomerular feedback by adenosine: evidence from mice lacking adenosine receptors. Proc Natl Acad Sci USA 98, 9983-9988, 2001; Nemeth, Z. H., et al. Adenosine receptor activation ameliorates type 1 diabetes. FASEB J, 2007; Chen, J. F., et al. A_{2A} adenosine receptor deficiency attenuates brain injury induced by transient focal ischemia in mice. J Neurosci 19, 9192-9200, 1999; Salvatore, C. A., et al. Disruption of the A₃ adenosine receptor gene in mice and its effect on stimulated inflammatory cells. The Journal of Biological Chemistry 275, 4429-4434, 2000). Animals were maintained in accordance with institutional and NIH guidelines under research protocols that were reviewed and approved by the University of Texas Health Science Center at Houston Animal Welfare Committee.

Blood Sample Collection and Preparation: Human: Four blood samples were withdrawn from a vein in the forearm of normal and SCD patients. Approximately 1 ml of blood was collected in 1.5 ml tube containing 10 μM dipyridamole and 10 μM 5′-deoxycoformycin (DCF) and the tubes were immediately immersed in liquid nitrogen and subsequently stored at −80° C. for metabolic analysis (e.g. adenosine). A 4 ml sample of blood was collected into a tube containing heparin as an anti-coagulant for measurement of 2,3-diphosphoglycerate (DPG) levels. A second 4 ml sample of blood was collected into a tube containing EDTA as an anti-coagulant and this sample was used for morphological studies, complete blood count (CBC) and hemoglobin electrophoresis. A 2 ml sample of blood was collected into a tube containing no anticoagulant for use in electrolyte and chemical panel assay.

Mouse: blood was collected in similar to human blood as described above except samples for ATP measurement, in which case blood was anticoagulated with stop solution.

Metabolomic Profiling. Unbiased metabolomic profiling of whole blood of control and SCD Tg mice was performed using liquid/gas chromatography coupled to mass spectrometry (LC/GC-MS) as described44. Specifically, a Thermofisher linear ion-trap mass spectrometer with Fourier transform and a Mat-95 XP mass spectrometer were used to analyze 251 named metabolites. The combinations of groups were analyzed using Welch's Two Sample t-test, following log transformation and imputation with minimum observed values for each compound. P<0.05 is considered significant.

Adenosine Analysis. The adenosine concentration present in whole blood was measured by HPLC as previously described (Knudsen, T. B., et al. Effects of (R)-deoxycoformycin (pentostatin) on intrauterine nucleoside catabolism and embryo viability in the pregnant mouse. Teratology 45, 91-103, 1992) with modifications. In brief, 500 μl of 0.6 M cold perchloric acid was added to 500 μl blood on ice, mixed using a vertex mixer and subsequently subject to sonication for 10 seconds with output 6 (W-220F, Heat Systems-Ultrasonic, Inc.). The homogenate was centrifuged at 20,000 g for 10 min at 40 C. The supernatant (568 μl) was transferred to a new tube and neutralized with 40.9 μl 3 M KHCO3/3.6 N KOH. Phenol red (2 μl of 0.2 mg/ml) was added as indicator. The sample was acidified with 5.7 μl of 1.8 M ammonium dihydrogen phosphate (pH 5.1) and 13.2 μl phosphoric acid (30%). Finally, the sample was centrifuged at 20,000 g for 5 min and the supernatant was transferred to a new tube and stored at −20° C. Before HPLC assay, the sample was thawed on ice, then centrifuged at 20,000 g for 10 minutes. The supernatant was transferred to a new tube for HPLC analysis as described previously (Aldrich, et al. Adenosine deaminase-deficient mice: models for the study of lymphocyte development and adenosine signaling. Adv Exp Med. Biol. 2000; 486, 57-63; Blackburn, et al. Metabolic and immunologic consequences of limited adenosine deaminase expression in mice. The Journal of Biological Chemistry. 1996; 271(25):15203-15210). Plasma adenosine measurements were performed as with whole blood, except the sonication step was omitted. Adenosine content was normalized to volume.

Chronic PEG-ADA therapy. SCD transgenic mice, at eight weeks of age, were divided to two groups. One group was injected with 2.5 U PEG-ADA weekly for 8 weeks. This dosing protocol was designated SCD with PEG-ADA treatment regimen (SCD+PEG-ADA). Another group was injected with normal saline for 8 weeks; it was designated as SCD without PEG-ADA treatment regimen (SCD). Wild-type mice (C57Bl/6) used as control were injected with saline or PEG-ADA as with the SCD groups.

Adenosine levels in the plasma of SCD Tg mice that received PEG-ADA treatment are presented in Supplementary FIG. 6. Adenosine levels were measured at different time points and the results indicated that weekly injection of PEG-ADA lowered adenosine to a steady level within two weeks of therapy.

Chronic treatment with A_{2B}R antagonist, PSB1115. SCD transgenic mice or wild type mice (eight weeks of age) were divided to two groups, one groups was injected with 200 μg of PSB1115 (an A_{2B} receptor antagonist: obtained from Tocris Bioscience, St. Louis, Mo.) in PBS, daily for 8 weeks. One of groups was injected with saline.

Hypoxia/reoxygenation of SCD Tg mice. SCD Tg mice were subjected to two-hour hypoxic condition with 8% oxygen. After hypoxia, mice were returned to normoxic conditions for four hours Wallace, et al. NKT cells mediate pulmonary inflammation and dysfunction in murine sickle cell disease through production of IFN-gamma and CXCR3 chemokines. Blood. 2009; 114(3):667-676). At the end of the experiments, mice were sacrificed and blood collected for adenosine measurement, sickling and hemolytic analysis.

Measurement of life span of RBCs in SCD Tg mice. SCD Tg mice were treated with or without PEG [symbol] ADA (2.5 unit/week) for 8 weeks. At 6 week-treatment, RBCs were labeled in vivo by using N-hydroxysuccinimide (NHS) biotin and the life span of circulating red blood cells was measured as described (Perumbeti, et al. A novel human gamma-globin gene vector for genetic correction of sickle cell anemia in a humanized sickle mouse model: critical determinants for successful correction. Blood. 2009; 114(6):1174-1185).

Specifically, 50 mg/kg of NHS biotin was injected into the retro-orbital plexus of SCD Tg mice (prepared in 100 μl sterile saline just prior to injection; initially dissolved at 50 mg/ml in N,—N,-dimethylacetamide) Blood samples (only 5 μl) were collected after first day of biotin-injection from tail vein by venipuncture to determine the percentage of RBCs labeled with biotin. Subsequently, 5 μl of blood were obtained by tail vein every 3 days for measurement of biotinylated RBCs until the 10th day. The percentage of biotinylated RBCs was calculated by determining the fraction of peripheral blood cells labeled with Ter-119 (to identify RBCs) that were also labeled with a streptavidin-conjugated fluorochrome by flow cytometry.

Isolation of total erythrocytes and treatment of human and mouse erythrocytes in vitro. Blood collected with heparin as an anti-coagulant was centrifuged at 240×g for 10 min at room temperature, followed by aspiration of plasma and white interface. Packed red blood cells (RBCs) were washed 3 times with culture media (F-10 Ham's with 1% penicillin/streptomycin, 10% fetal bovine serum (FBS), and re-suspended to 4% hematocrit (HCT). One ml of RBCs was added to each well of a 12-well plate. Normal RBCs purified from humans were treated with different concentrations of 5′-(N-ethyl-carboxamido) adenosine (NECA, an adenosine receptor agonist, Sigma-Aldrich, St. Louis, Mo.) ranging from 1-50 μM for different time points (from 1 to 18 hr) under normoxic conditions. Normal RBCs were treated with 10 μM NECA in the presence or absence of adenosine receptor antagonists, including theophylline, PSB36, SCH442416, MRS1754 and MRS3777 (Tocris Bioscience, St. Louis, Mo.) at 10 μM. In addition, normal RBCs were treated directly with the A_{2B}R agonist (BAY 60-6583, Bayer HealthCare AG, Wuppertal, Germany) or the A_{2A}R agonist (CGS21680) (Tocris). Similarly, the mouse RBCs were treated with or without NECA at 10 μM in the presence or absence of theophylline (10 μM). In addition, RBCs purified from 4 adenosine receptor deficient mice were treated with 10 μM NECA. After 18 hours, cells were collected and 2,3-DPG levels were measured as described below. RBCs were purified from SCD patients and treated with or without 10 μM NECA, 5 units/ml polyethylene glycol-adenosine deaminase (PEG-ADA) or 3 mmol/L glycolic acid (Sigma-Aldrich), 10 μM MRS1754 (A_{2B}R antagonist) under ambient oxygen (normoxia) or different levels of hypoxic conditions (for 3 hours with shaking at 37° C. At the end of experiments, 2,3-DPG levels were measured and the percentage of sickled cells determined as described below.

Morphological study of erythrocytes and quantification of reticulocytes by flow cytometry. Blood smears were stained using the WG16-500 ml kit (Sigma-Aldrich) for sickled cells and observed using 100× oil immersion objective of Olympus BX60 microscope. Areas where red blood cells do not overlap were randomly picked, at least 10 fields were observed and 1000 red blood cells including irreversible sickled cells were counted. The percentages of sickled cells among the counted cells were calculated. Reticulocyte was labeled by Retic-COUNT Reagent (Becton Dickinson) and quantified by flow cytometry (Perumbeti, et al., A novel human gamma-globin gene vector for genetic correction of sickle cell anemia in a humanized sickle mouse model: critical determinants for successful correction. Blood. 2009; 114(6):1174-1185).

Mouse urine collection and measurements. Urine was collected using a metabolic cage (Bioseb, France) for proteinuria and creatinine analysis using a commercially available kit (Exocell, Philadelphia) as described Zhou, et al. Angiotensin receptor agonistic autoantibodies induce pre-eclampsia in pregnant mice. Nat. Med. 2008; 14(8):855-862). For urine osmolality analysis, mice were deprived of water for 24 hours, then urine was collected on Parafilm, transferred to 1.5 ml tube, the urine was diluted 10 times with distilled water and measured with vapor pressure osmometer (Wescor, Logan, Utah).

Mouse organ isolation and histological analysis. Mice were anesthetized and body weight was determined. Organs were isolated and weighed. Half of each organ was quickly frozen in liquid nitrogen and then stored at −80° C. for heme content measurements as described below. The remaining half of each organ was fixed with 4% paraformaldehyde in PBS overnight at 4° C. Fixed tissues were rinsed in PBS, dehydrated through graded ethanol washes, and embedded in paraffin. 5 μm sections were collected on slides and stained with haematoxylin and eosin (H&E). Semiquantification of histological changes was analyzed by Image-Plus, Pro software.

Heme content measurement in multiple mouse tissues. Different organs were quickly removed and frozen in liquid nitrogen as described above. Heme was extracted from organs by homogenization with 1% Triton x-100 in PBS with 1× proteinase inhibitors (Roche) and the supernatant was collected following centrifugation at 20,000 g for 10 minutes at 4° C. Heme content was quantified following the instructions of QuantiChrom Heme Assay Kit (BioAssay Systems, Hayword, Calif.).

2,3-DPG analysis. 2,3-DPG concentration in RBCs was detected by a commercially available kit (Roche, Nutley, N.J., and as described in Ericson A, de Verdier C H. A modified method for the determination of 2,3-diphosphoglycerate in erythrocytes. Scandinavian journal of clinical and laboratory investigation. 1972; 29(1):84-90).

Hemolytic analysis. The hemoglobin, haptoglobin and total bilirubin in mouse plasma were quantified by ELISA kits following instructions provided by the vendor (BioAssay Systems, Hayword, Calif.).

Measurement of percentage of saturated Hb in mice. Percentage of saturated Hb (SpO2) in mice was measured by Oxysat (Kent Scientific Corp, CT). Mice were held in mouse holders (Kent Scientific Corp, CT) and Oxysat sensor was placed on a site approximately 1 cm from the base of the tail. When mouse was calm, the SpO2 was continuously recorded for about 2 minutes. The data were collected and SpO2 was calculated by average of SpO2 in two-minute measurement.

Immunofluorecent staining of A_{2B}R adenosine receptor. Human or mouse blood smears were fixed with 100% cold methanol for 10 minutes at room temperature, then incubated with de-ionized water for 10 minutes, blocked by 10% FBS, 1% BSA in PBS (pH7.4) for one hour at room temperature. The slide was incubated with 40 μg/ml anti-A_{2B} receptor antibody (Millipore, Billerica, Mass.) in blocking buffer overnight at 40 C, washed with PBS 3×, incubated with donkey anti-rabbit IgG (H+L)-568 (Invitrogen, Carlsbad, Calif.) for one hour at room temperature in the dark. The slide was washed 3×, then mounted on cover glass with mounting medium (VECTASHIELD H-1200, Vector Llabs Burlingame, Calif.). Pictures were taken under Zeiss LSM 510 confocal microscope (Carl Zeiss Inc, Jena, Germany).

Immunohistochemistry to assess neutrophil infiltration in the lung. To quantify neutrophils, a specific neutrophil antibody, was used as described (Zhou, et al., Enhanced airway inflammation and remodeling in adenosine deaminase-deficient mice lacking the A_{2B} adenosine receptor. J. Immunol. 2009; 182(12):8037-8046). Rehydrated slides were quenched with 3% hydrogen peroxide, Ag retrieval performed (Dako), and endogenous avidin and biotin blocked with a Biotin Blocking System (Dako). Slides were incubated with a rat anti-mouse neutrophil Ab (AbD SeroTec, 1/500 dilution, overnight at 4° C.). Sections were incubated with ABC Elite Streptavidin reagents and appropriate secondary Abs, then developed with 3,3′-diaminobenzidine (Sigma-Aldrich), and counterstained with methyl green. Quantification of distal airway neutrophils-positive cells was performed on 20 fields/mouse lung section at ×20 using software analysis (Image Pro Plus 4.0; Media Cybernetics, Bethesda, Md., USA).

ELISA for INF-γ, IL-6, IL-1β and GM-CSF measurement in the mouse lung homongenates. INF-γ, IL-6, IL-1β and GM-CSF levels in lung homogenates were determined using ELISA kits (QUANGI).

cAMP measurement. Quantitative assays for cAMP contents in RBCs were performed by using a commercial enzyme immunoassay kit (Assay Designs Inc., Michigan, USA).

Plasma ADA activity measurement. Plasma was isolated as described above and centrifugated at 4000×g for 10 min at 4° C. ADA activity was measured in the supernatants obtained from high-speed centrifugation under saturating substrate conditions using a spectrophotometric assay as described (Blackburn M R, Datta S K, Kellems R E. Adenosine deaminase-deficient mice generated using a two-stage genetic engineering strategy exhibit a combined immunodeficiency. Journal of Biological Chemistry. 1998; 273(9):5093-5100). The decrease in absorbance at 265 nm resulting from deamination of adenosine to inosine was continuously monitored in a Beckman DU-50 spectrophotometer and the rate of inosine production was calculated at linearity. Specific activities are presented as nanomoles of adenosine converted to inosine per min per mg of protein.

The ADA activity in the plasma of WT and SCD Tg mice was analyzed and the results indicated that ADA activity in the plasma of SCD Tg was not significantly different from that of WT mice (Supplementary FIG. 5).

Statistical analysis. All data were expressed as the mean±SEM. Data were analyzed for statistical significance using GraphPad Prism 4 software (GraphPad Software, San Diego, Calif.). Student's t tests (paired or unpaired as appropriate) were applied in two-group analysis. Differences between the means of multiple groups were compared by the one-way analysis of variance (ANOVA), followed by a Tukey's multiple comparisons test. A value of P<0.05 was considered significant and was the threshold to reject the null hypothesis.

Plasma ATP measurement. The blood was collected in a centrifuge tube containing anticoagulant in a “stop solution” as described (Gorman, et al., Measurement of adenine nucleotides in plasma. Luminescence. 2003; 18(3):173-181). The plasma was isolated by centrifugation at 2000 g for 1 min. Then, 50 μl of plasma was added to 150 μl 0.6 M cold perchloric acid on ice, vortexed and centrifuged at 20,000 g for 10 min at 40 C. 100 μl supernatant was transferred to a new tube and neutralized with 50 μl 0.6 N KHCO3/0.72N KOH. Finally, the sample was centrifuged at 20,000 g for 5 min and the supernatant was transferred to a new tube and stored at −200 C for ATP measurement by luciferase assay (Gorman, et al., 2003, ibid).

ATP levels in the plasma of WT and SCD Tg mice were analyzed and the results indicated that ATP concentrations in plasma from SCD Tg mice were significantly increased compared with that from WT mice (Supplementary FIG. 4).

### Example 1

### Adenosine is Highly Elevated in the Blood of SCD Tg Mice

Metabolomic profiling revealed that adenosine was among the most highly elevated metabolites in the whole blood of SCD Tg mice compared to controls (Supplementary FIG. 1). Next, it was determined that adenosine was also significantly elevated in the plasma of SCD Tg mice (FIGS. 1a & b). Adenosine is an endogenous nucleoside known to increase under hypoxic conditions due to the degradation of extracellular ATP from affected cells or tissues and functions as a ligand to activate four adenosine receptors.

### Example 2

### Elevated Adenosine Contributes to Chronic Sickling Seen in SCD

To establish that increased adenosine contributes to sickle cell disease in vivo we treated SCD Tg mice with polyethylene glycol-modified adenosine deaminase (PEG-ADA), a drug that has been successfully used for over twenty years to successful lower adenosine concentrations in ADA-deficient humans (FIG. 1b). Following an 8 week regimen of PEG-ADA treatment, blood smear analysis and flow cytometric analysis of reticuclotyes revealed that the percentages of sickled RBCs and reticulocytes were significantly reduced (FIG. 1c and Table 1). This indicates that increased adenosine contributes to chronic sickling in SCD Tg mice.

Erythrocyte sickling is the primary cause of intravascular hemolysis that accompanies SCD and unexpectedly it was also determined that intravascular hemolysis in SCD Tg mice was significantly reduced by PEG-ADA treatment as demonstrated by decreased plasma Hb, increased plasma haptoglobin, and decreased total bilirubin (FIG. 1d-f). It was determined that the half-life of RBCs in SCD Tg mice increased from 2 days to 4 days (2 fold increase) with chronic PEG-ADA treatment (FIG. 1g). Complete blood count (CBC) analysis showed that chronic PEG-ADA treatment significantly increased the total number of RBCs, Hb concentration, hematocrit (HCT) and lowered the total number of white blood cells (WBCs) (Table 1). The increase in hematocrit (Table 1) presumably reflects the corresponding increase in RBC numbers. Of note, we found that red cell distribution width (RDW) was also significantly reduced by PEG-ADA treatment (Table 1), suggesting that the sizes of RBCs were more uniform and regular. Altogether, these studies demonstrate that decreased sickling, hemolysis and prolonged life span of RBCs with PEG-ADA treatment result in significantly increased erythrocyte number, total hemoglobin content and a decreased inflammatory response in SCD Tg mice.

### Example 3

### Reduction in Adenosine with PEG-ADA Therapy Reduces Tissue Damage and Dysfunction in SCD

In addition to resulting in hemolysis, RBC sickling also led to multiple tissue damage in SCD Tg mice. Histological analysis revealed that the congestion, vascular damage and necrosis common in multiple tissues (including lung, liver and spleen) of SCD Tg mice were also improved by chronic treatment with PEG-ADA (FIG. 2a). Of note, the ratio of spleen weight to body weight was reduced from 4.52±0.24 to 2.48±0.36 following chronic PEG-ADA treatment (p<0.05, n=8).

SCD Tg mice, with and without PEG-ADA treatment, were compared in a semi-quantitative analysis of histological changes. This analysis revealed a significant degree of improvement with regards to pathological changes associated with SCD following PEG-ADA treatment (FIG. 2b-d).

Supporting these histological improvements in the tissue of SCD Tg mice following PEG-ADA enzyme therapy, significantly decreases in the normally elevated heme content were observd in all the tissues we examined including lung, liver and spleen in SCD Tg mice (FIG. 2e-g).

Both SCD Tg mice and approximately 25 percent of patients with SCD develop renal dysfunction with proteinuria. It was established that the increased microinfarction and cysts seen in the renal cortex and the congestion in the renal medulla normally observed in SCD Tg mice were significantly improved by administration of PEG-ADA enzyme therapy. (FIG. 2h). Chronic PEG-ADA enzyme therapy had no significant effect on the control mice (FIG. 2h). Semiquantification of histological changes indicated that PEG-ADA treatment significantly improved the renal damage in SCD Tg mice (FIG. 2i-j). Correlating with the improved histological appearance of kidneys following PEG-ADA therapy, we found that chronic PEG-ADA enzyme therapy also decreased proteinuria and increased urine osmolality, features indicating improvement of kidney function in SCD Tg mice chronically treated with PEG-ADA (FIG. 2k-l). These studies provide additional evidence supporting the detrimental role of excess adenosine in the pathophysiology associated with SCD and the beneficial effects of reducing adenosine levels using chronic PEG-ADA enzyme therapy.

### Example 4

### Reduction in Adenosine with PEG-ADA Therapy Moderates Symptoms of Acute Sickle Crisis

It is well-established that hypoxia/reoxygenation triggers an acute sickle crisis in SCD. To establish the role of adenosine and the ability of compounds that reduce adenosine levels to prevent some of the symptoms associated with SCD crisis, SCD Tg were exposed to 2 hour of hypoxia followed by 4 hours of reoxygenation (2 h hypoxia/4 h reoxygenation) and as expected already elevated plasma adenosine levels were raised further as compared to steady state levels in SCD Tg mice under normoxic conditions (FIG. 3a). However, pretreatment with PEG-ADA prior to hypoxia/reoxygenation resulted in significantly lowered adenosine levels (FIG. 3a), reduced sickling (FIG. 3b) and attenuated hemolysis, as indicated by decreased plasma hemoglobin (FIG. 3c) and plasma total bilirubin (FIG. 3d). Consistently, complete blood counts (CBC) also showed that PEG-ADA pre-treatment significantly prevented hypoxia/reoxygenation-induced decrease in RBCs, Hb, HCT and also attenuated the further increase in RDW and WBC that usually occurs in these mice under these conditions (Supplementary Table 1). These findings provide strong evidence that elevated adenosine is responsible for the increased erythrocyte sickling and hemolysis that follows an acute sickle crisis event and that preventing the rise in or lowering adenosine levels can prevent many of the acute crisis symptoms from occurring.

In addition to sickling and hemolysis, vaso-occlusion is a key endpoint of an acute crisis event with SCD. To establish the effect of PEG-ADA therapy on vaso-occlusion, we chose to assess hypoxia-reoxygenation-induced lung inflammation, a well accepted measurements of vaso-occlusion. Immunostaining with neutrophil specific antibody demonstrated that hypoxia-reoxygenation increased neutrophil infiltration in the lungs of SCD Tg mice compared to normoxic conditions (FIG. 3e). PEG-ADA treatment reduced neutrophil infiltration in the lungs following hypoxia-reoxygenation conditions (FIG. 3e). Image quantification analysis confirmed that PEG-ADA treatment significantly lowered hypoxiareoxygenation-induced neutrophil infiltration (FIG. 3f). Consistently, PEG-ADA treatment significantly decreased the levels of a series of proinflammatory cytokines in the lungs of SCD Tg mice, including INF-γ, IL-6, IL-1β and GM-CSF (FIG. 4g). Thus, these findings indicate that elevated adenosine not only underlies hypoxia-reoxygenation-induced sickling, but also contributes to hypoxia-reoxygenation-induced lung inflammation, a major outcome of vaso-occlusion in acute sickle crisis.

### Example 5

### Adenosine Levels act Through the 2,3-diphosphorglycerate pathway

To identify and characterize the intracellular mediators induced by increased adenosine levels that result in increased RBC sickling, a number of metabolites that were differentially present in RBCs from SCD Tg mice or from controls mice, prior to and following treatment with PEG-ADA for 8 weeks were examined.

It was determined that 2,3-diphosphoglycerate (2,3-DPG, also called 2,3-bisphosphoglycerate), an erythrocyte specific byproduct of glycolysis, was increased in whole blood of SCD Tg mice (Supplementary FIG. 1). It was confirmed that 2,3-DPG levels were significantly elevated in the RBCs from SCD Tg mice at steady state under normoxic conditions (FIG. 4a). Previous studies have shown that 2,3-DPG is an erythrocyte specific metabolite known to reduce the oxygen binding affinity of Hb. Early studies indicate that 2,3-DPG levels are increased in the RBCs of SCD patients and contribute to erythrocyte sickling under hypoxic conditions. Because adenosine was elevated in steady state (FIG. 1b) and responsible for sickling, it is possible that elevated 2,3-DPG seen in SCD Tg mice is a mediator of adenosine-induced sickling Supporting this hypothesis lowering adenosine levels in SCD Tg mouse at steady state with chronic PEG-ADA treatment resulted in a reduction of 2,3-DPG levels in the RBCs (FIG. 4a), increased Hb binding affinity featured by increased percentage of saturated Hb (FIG. 4b) and attenuated chronic sickling (FIG. 1c & Table 1). Similarly, it was determined that 2,3-DPG was further elevated by hypoxia/reoxygenation and that pretreatment with PEG-ADA significantly inhibited its elevation (FIG. 4c), indicating that elevated adenosine is responsible for hypoxia/reoxygenation-mediated 2,3-DPG induction. Significantly,

Hypoxia/reoxygenation-induced acute vascular crisis which is usually accompanied by remarkable increases in sickling, hemolysis and pulmonary inflammation was prevented by PEG-ADA pretreatment (FIG. 3b-d). Thus, these results indicate that adenosine is a previously unrecognized factor responsible for increased 2,3-DPG in erythrocytes of SCD Tg mice and suggest that elevated 2,3-DPG contributes to both chronic sickling and acute sickle crisis.

### Example 6

### Adenosine-Mediated Signaling Through the A_{2B} Adenosine Receptor

In order to further characterize factors responsible for adenosine induced erythrocyte sickling metabolic profiles of control and SCD mice, with a particular focus on erythrocyte specific metabolites. Next, to determine whether adenosine can directly induce 2,3-DPG levels in RBCs, we treated primary normal mouse mature erythrocytes with 5′-N-ethylcarboxamidoadenosine (NECA), a potent, non-metabolizable adenosine analog. It was determined that NECA stimulated an increase in 2,3-DPG levels (FIG. 4d), clearly indicating that adenosine can directly induce 2,3-DPG levels in mature mouse RBCs.

Adenosine is a potent signaling molecule that elicits many physiological and pathological effects by activation of G-protein coupled receptors on target cells. Four such receptors have been identified, A₁R, A_{2A}R, A_{2B}R and A₃R, each having a unique affinity for adenosine and a distinct cellular and tissue distribution. Thus, to characterize whether adenosine-mediated 2, 3-DPG induction seen in erythrocytes occurs through adenosine receptor signaling, normal mouse RBCs were treated with NECA in the presence or absence of theophylline, a broad spectrum adenosine receptor antagonist. It was determined that NECA-mediated 2,3-DPG induction was prevented by the presence of theophylline, a broad spectrum adenosine receptor antagonist (FIG. 4d). To establish which adenosine receptor was responsible for NECA-mediated 2,3-DPG induction, RBCs purified from four different adenosine receptor-deficient mice, were used. It was determined that RBCs purified from A₁R, A_{2A}R and A₃R deficient mice and WT mice showed a similar 2,3-DPG induction following treatment with NECA. In contrast, NECA-mediated 2,3-DPG induction did not occur with RBCs from A_{2B}R-deficient mice (FIG. 4e). Supporting this finding, immunostaining with A_{2B}R specific antibody revealed that A_{2B}R was expressed in isolated mouse erythrocytes from wild type mice but not A_{2B}R deficient mice (FIG. 4f). Finally, it was determined that hypoxia-mediated 2,3-DPG induction was also significantly decreased in the RBCs from A_{2B}R-deficient mice (FIG. 4g). Altogether these results provide strong genetic evidence that A_{2B}R signaling is required for hypoxia-mediated 2,3-DPG induction in normal mouse RBCs. The A_{2B}R is commonly coupled to adenylyl cyclase by the stimulatory G-protein subunit (Gas) and serves to increase intracellular cAMP resulting in the activation of PKA.

In order to characterize the role of cAMP-dependent PKA activation in A_{2B}R-mediated induction of 2,3-DPG, it was determined that NECA was capable of inducing cAMP production in RBCs from WT mice, but not A_{2B}R-deficient mice (FIG. 4h). Moreover, it was determined that t treatment of normal mouse RBCs with H-89, a specific potent PKA inhibitor, significantly inhibited NECA-mediated 2,3-DPG induction (FIG. 4i). These findings indicate that that A_{2B}R-mediated cAMP-dependent PKA activation is responsible for adenosine-mediated 2,3-DPG induction in normal RBCs.

### Example 7

### In Vivo Effects of A_{2B}R Antagonism

To determine the in vivo significance of A_{2B}R-mediated 2,3-DPG induction on sickling in SCD Tg mice, the mice were treated for 8 weeks with the A_{2B}R specific antagonist, PSB1115. As with PEG-ADA treatment (FIG. 4a), treatment with PSB1115 resulted in decreases in 2, 3-DPG levels (FIG. 4i) and the percentage of the sickled cells (Table 1). Moreover, it was determined that PSB1115 chronic treatment increased the half-life of RBCs from SCD Tg mice from 2 days to 5.5 days (a 2.75 fold increase) (FIG. 4k), which is slightly higher than the improvement observed in PEG-ADA-treated mice (FIG. 1g). In addition, CBC analysis showed significant improvements with PSB1115 treatment, including increased total RBCs, total Hb, decreased total WBCs, reticulocytes and RDW (Table 1). Similar to chronic PEG-ADA enzyme therapy, histological studies showed that chronic treatment with PSB1115 reduced congestion, vascular damage and necrosis in multiple tissues, including lung, liver, spleen and kidney (Supplementary FIG. 2a). Image quantification analysis showed the improvement of tissue injury in SCD Tg mice by PSB1115 chronic treatment was significant (Supplementary FIG. 2b-f). Overall, these studies provide in vivo evidence that A_{2B}R-mediates 2,3-DPG induction and erythrocyte sickling and subsequently multiple organ damage in SCD.

### Example 8

### A_{2B}R-Mediated Adenosine-Induced Sickling in Erythrocytes of Human Scd Patients

Human Subjects: Sickle cell disease patients in the steady state were identified by a hematologist on the faculty of the University of Texas Medical School at Houston. Patients participating in this study had no blood transfusion for at least 6 months before blood samples were collected. Half of the patients were treated with hydroxyurea. Control subjects were of African descent and were free of hematological diseases. Relevant clinical features of study patients are presented in Supplementary Table 2. The research protocol, which included informed consent from the subjects, was approved by the University of Texas Health Science Center at Houston Committee for the Protection of Human Subjects.

Blood collection and hematological analysis. Approximately 1 ml of blood was withdrawn from a forearm vein of normal individuals and SCD patients. The blood was collected in 1.5 ml tube containing 10 μM dipyridamole, 10 μM α,β-methylene ADP and 10 μM 5′-deoxycoformycin (DCF), immediately dropped into liquid nitrogen and subsequently stored at −80° C. for metabolic analysis. 4 ml of blood was collected with heparin as an anti-coagulant for 2, 3-diphosphoglycerate (2,3-DPG) measurement. An additional 4 ml of blood was collected with EDTA as an anti-coagulant and used for morphological study, complete blood count (CBC) and hemoglobin electrophoresis and 1 ml of blood was aliquoted to 1.5 ml tubes containing 10 μM dipyridamole and 10 μM DCF for plasma adenosine assay.

To demonstrate the patho-physiological significance of adenosine signaling in humans with SCD, adenosine and 2,3-DPG levels were measured in the blood of both control human subjects and SCD patients (for patient information see Supplementary Table 2). HPLC results showed that the adenosine concentration was elevated in the blood of SCD patients compared to controls (FIG. 5a). Consistent with the finding described in the previous examples and in vivo findings from SCD Tg mice (FIG. 4a), it was determined that the concentration of 2,3-DPG was significantly elevated in RBCs of SCD patients compared to that of normal individuals (FIG. 5b). It was also determined that the A_{2B}R is expressed on human erythrocytes (Supplementary FIG. 3a) and that NECA stimulated 2,3-DPG levels in a dosage and time-dependent manner in primary cultured human RBCs from normal individuals (Supplementary FIGS. 3b & 3c). NECA-mediated 2,3-DPG induction was inhibited by theophylline, indicating the requirement for adenosine receptor signaling (Supplementary FIG. 3d). Further predicted by the finding in SCD Tg mice, it was determined that NECA-mediated 2,3-DPG induction in RBCs from normal individuals was inhibited only by the A_{2B}R antagonist (MRS1754) but not other adenosine receptor (AR) antagonists (including A₁R, A_{2A}R and A₃R antagonists (PSB36, SCH442416 and MRS3777, respectively) (Supplementary FIG. 3d). In agreement with these findings the A_{2B}R agonist (BAY 60-6583) but not the A_{2A}R agonist (CGS21680) induced 2,3-DPG production in a dosage-dependent manner in RBCs (Supplementary FIGS. 3e & 3g). Thus, similar to the SCD Tg mouse model, the A_{2B}R is required for adenosine-mediated 2,3-DPG induction in normal human RBCs.

### Example 9

### Antagonists of A_{2B}R Prevent Adenosine-Induced Sickling in Erythrocytes of Human Scd Patients

To establish the pathophysiological significance of adenosine-mediated 2,3-DPG in erythrocyte sickling in humans, primary erythrocytes purified from SCD patients under hypoxic conditions (2% oxygen) to induce sickling in the presence or absence of PEG-ADA (reduces adenosine levels), NECA (non-metabolizable adenosine analog), MRS1754 (interferes with A_{2B}R signaling), H-89 (PKA specific inhibitor) or glycolate (GA, depletes 2,3-DPG) were determined. At the end of the studies, 2,3-DPG levels were determined. It was determined that hypoxic conditions resulted in the induction of 2,3-DPG (FIG. 5c). Next, it was determined that PEG-ADA, MRS1754, H-89 and glycolate reduced hypoxia-mediated induction of 2,3-DPG to a similar extent (FIG. 5c), indicating that adenosine is responsible for 2,3-DPG induction via A_{2B}R signaling. In contrast, NECA significantly stimulated a further increase in 2,3-DPG levels under hypoxic conditions (FIG. 5c). These findings reveal that A_{2B}R-mediated PKA activation is required for hypoxia-induced 2,3-DPG induction in human sickle erythrocytes.

Finally, to determine the functional role of A_{2B}R-mediated 2,3-DPG induction in sickling, RBCs from individuals with SCD were cultured under different oxygen pressures and in the presence or absence of NECA, PEG-ADA, MRS1754, H-89 or GA. It was determined that the percentage of sickle cells was inversely dependent on oxygen concentration (FIG. 5d). Treatment with PEG-ADA, MRS1754, H-89 or GA significantly reduced the percentage of sickled cells, while NECA significantly increased the percentage of sickled cells under hypoxic conditions (FIG. 5d). Overall, these findings demonstrate that excess adenosine-mediated 2, 3-DPG induction via A_{2B}R-mediated PKA activation is a major underlying mechanism contributing to hypoxia-mediated erythrocyte sickling in RBCs isolated from patients with SCD.

In total the findings described in the present disclosure reveal that increased adenosine is a previously unrecognized regulator of 2,3-DPG induction and that it acts through the A_{2B}R receptor responsible for SCD associated symptoms and disorders, particularly but are not limited to, the sickling of erythrocytes, oxygen release, increased hemoglobin (HbS) polymerization, hemolysis, tissue congestion and disruption and organ damage or failure in mammals such as humans, mice and companion animals. Thus the present disclosure identifies new methods and molecular targets as well as compositions directed at interfering with adenosine signaling, by among other things applying A_{2B}R antagonist therapies as an effective method of treating SCD and its associated disorders in man.

Without further elaboration, it is believed that one skilled in the art can, using the description herein, utilize the present invention to its fullest extent. The embodiments described herein are to be construed as illustrative and not as constraining the remainder of the disclosure in any way whatsoever. While the preferred embodiments have been shown and described, many variations and modifications thereof can be made by one skilled in the art without departing from the spirit and teachings of the invention. For example, although the described embodiments illustrate use of the present compositions and methods on humans, those of skill in the art would readily recognize that these methods and compositions could also be applied to veterinary medicine and other mammals. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

TABLE 1
Hematological parameters of WT and SCD Tg mice treated with or without
either PEG-ADA or PSB1115 for 8 weeks

| | RBC | Hb | HCT | MCV | MCH |
|---|---|---|---|---|---|
| | (M/μl) | (g/dl) | (%) | (fl) | (pg) |
| WT (untreated) | 8.87 ± 0.307 | 13.75 ± 0.87 | 45.15 ± 2.44 | 49.47 ± 0.57 | 15.05 ± 0.23 |
| (n = 4) | | | | | |
| WT (+PEG-ADA) | 8.81 ± 0.18 | 13.28 ± 0.67 | 43.83 ± 1.16 | 49.75 ± 0.54 | 15.07 ± 0.46 |
| (n = 4) | | | | | |
| WT (+PSB1115) | 8.81 ± 0.39 | 13.28 ± 0.54 | 45.26 ± 1.64 | 49.12 ± 0.80 | 14.4 ± 0.29 |
| (n = 4) | | | | | |
| SCD (untreated) | 4.27 ± 1.08* | 5.05 ± 1.10* | 20.70 ± 4.16* | 49.88 ± 8.65 | 12.08 ± 1.96* |
| (n = 8) | | | | | |
| SCD (+PEG-ADA) | 6.15 ± 1.01** | 7.71 ± 2.64** | 30.15 ± 8.90** | 48.58 ± 8.79 | 12.38 ± 2.85 |
| (n = 8) | | | | | |
| SCD (+PSB1115) | 6.45 ± 0.45** | 8.25 ± 0.29** | 31.82 ± 3.43** | 49.17 ± 1.82 | 12.78 ± 0.46 |
| (n = 6) | | | | | |
| | MCHC | RDW | Reticulocyte | Sickle cell | WBC |
| | (g/dl) | (%) | (%) | (%) | (k/μl) |
| WT (untreated) | 30.47 ± 0.65 | 15.05 ± 0.63 | ND | ND | 3.25 ± 0.95 |
| (n = 4) | | | | | |
| WT (+PEG-ADA) | 30.27 ± 0.79 | 15.05 ± 0.75 | ND | ND | 3.27 ± 0.81 |
| (n = 4) | | | | | |
| WT (+PSB1115) | 29.34 ± 0.41 | 14.1 ± 0.67 | ND | ND | 3.89 ± 1.52 |
| (n = 4) | | | | | |
| SCD (untreated) | 24.31 ± 1.47* | 31.61 ± 0.63* | 62 ± 4.58 | 18.29 ± 0.59 | 26.03 ± 8.49* |
| (n = 8) | | | | | |
| SCD (+PEG-ADA) | 25.33 ± 1.21 | 28.03 ± 1.14** | 43 ± 4.52** | 10.35 ± 0.39** | 15.00 ± 2.79** |
| (n = 8) | | | | | |
| SCD (+PSB1115) | 26.02 ± 1.82 | 28.27 ± 1.36** | 37.15 ± 3.27** | 9.07 ± 0.75** | 11.95 ± 3.27** |
| (n = 6) | | | | | |

WT: Wild type;
SCD: sickle cell disease transgenic mice;
RBC: red blood cells;
Hb: hemoglobin;
HCT: hematocrit;
MCV: mean corpuscular volume;
MCH: mean corpuscular hemoglobin;
MCHC: mean corpuscular hemoglobin concentration;
RDW: red cell distribution width;
WBC: white blood cell.
*P < 0.05 vs. WT and
**P < 0.05 vs. SCD Tg mice without treatment.

SUPPLEMENTARY TABLE 1
Complete blood count in SCD Tg mice treated with or without PEG-ADA under hypoxic/reoxygenation condition

| | RBC | Hb | HCT | MCV | MCH | MCHC | RDW | WBC |
|---|---|---|---|---|---|---|---|---|
| Mice | (M/μl) | (g/dl) | (%) | (fl) | (pg) | (g/dl) | (%) | (k/μl) |
| SCD (untreated) | 3.29 ± 0.45 | 3.05 ± 0.36 | 16.72 ± 4.1 | 50.87 ± 8.36 | 9.10 ± 1.70 | 17.89 ± 0.49 | 33.3 ± 3.20 | 33.3 ± 6.20 |
| n = 5 | | | | | | | | |
| SCD + PEG-ADA | 4.94 ± 0.17* | 4.58 ± 0.38* | 21.25 ± 2.52* | 48.93 ± 2.05 | 9.12 ± 0.72 | 18.65 ± 1.82 | 29.72 ± 1.95* | 18.68 ± 3.32* |
| n = 7 | | | | | | | | |

SCD: sickle cell disease transgenic mice;
RBC: red blood cells;
Hb: hemoglobin;
HCT: hematocrit;
MCV: mean corpuscular volume;
MCH: mean corpuscular hemoglobin;
MCHC: mean corpuscular hemoglobin concentration;
RDW: red cell distribution width;
WBC: white blood cell.
*P < 0.05 vs. SCD Tg mice without treatment.

SUPPLEMENARY TABLE 2
Information of control individuals and SCD patients

| | Control | SCD |
|---|---|---|
| Number | 12 | 14 |
| Gender | M = 3 F = 9 | M = 6 F = 8 |
| Age (years) | 43 ± 3.45 | 34 ± 3.05 |
| RBC (10⁶/μl) | 4.645 ± 0.437 | 2.779 ± 0.682* |
| Hb (g/dl) | 13.241 ± 1.197 | 8.864 ± 1.868* |
| HCT (%) | 40.558 ± 3.932 | 26.086 ± 5.508* |
| WBC (10³/μl) | 5.592 ± 1.472 | 10.614 ± 4.084* |
| Hgb S (%) | NT | 70.233 ± 19.165 |
| Hgb A (%) | NT | 8.782 ± 13.939 |
| Hgb A₂ (%) | NT | 4.108 ± 0.871 |
| Hgb F (%) | NT | 14.208 ± 14.389 |

NT: not tested
*P < 0.05 vs. Control

What is claimed is:

1. A method of treating sickle cell disease, comprising:
administering to a person suffering from sickle cell disease a composition comprising an effective amount of at least one inhibitor of adenosine signaling and a pharmaceutically-acceptable carrier, wherein the inhibitor of adenosine signaling has at least one activity selected from the group consisting of decreasing adenosine levels in the mammal, inhibiting adenosine receptor activity in the mammal, and inhibiting signaling pathways downstream of an adenosine receptor in the mammal.

2. The method of claim 1, wherein the at least one inhibitor of adenosine signaling is selected from the group consisting of adenosine deaminase (ADA), polyethylene-glycol modified adenosine deaminase (PEG-ADA), 5′-nucleotidase inhibitors, theophylline, adenosine receptor A_{2B} antagonists, adenylyl cyclase inhibitors, protein kinase A inhibitors, bisphosphoglycerate mutase inhibitors, glycolate, and salts and esters thereof.

3. The method of claim 1, wherein the at least one inhibitor of adenosine signaling is an antagonist of the A_{2B} adenosine receptor.

4. The method of claim 1, wherein said antagonist of the A_{2B} adenosine receptor is drawn from the group consisting of theophylline, PSB36, SCH442416, MRS1754 and MRS3777.

5. The method of claim 1, wherein administering comprises one or more routes of administration selected from the group consisting of intravenous administration, intraperitoneal administration, intramuscular administration, intradermal administration, oral administration, and transdermal administration.

6. A kit for treating sickle cell disease, comprising at least one inhibitor of adenosine signaling, a pharmaceutically-acceptable carrier, and instructions for the method of claim 1.

7. The kit of claim 6, wherein the at least one inhibitor of adenosine signaling is selected from the group consisting of adenosine deaminase (ADA), polyethylene-glycol modified adenosine deaminase (PEG-ADA), 5′-nucleotidase inhibitors, theophylline, adenosine receptor A_{2B} antagonists, adenylyl cyclase inhibitors, protein kinase A inhibitors, bisphosphoglycerate mutase inhibitors, glycolate, and salts and esters thereof.

8. A method of manufacturing a medicament for the treatment of sickle cell disease, comprising:
combining an effective amount of at least one inhibitor of adenosine signaling and a pharmaceutically-acceptable carrier.

9. The method of claim 8, wherein the at least one inhibitor of adenosine signaling is selected from the group consisting of adenosine deaminase (ADA), polyethylene-glycol modified adenosine deaminase (PEG-ADA), 5′-nucleotidase inhibitors, theophylline, adenosine receptor A_{2B} antagonists, adenylyl cyclase inhibitors, protein kinase A inhibitors, bisphosphoglycerate mutase inhibitors, glycolate, and salts and esters thereof.

10. The method of claim 8, wherein the at least one inhibitor of adenosine signaling is an antagonist of the A_{2B} adenosine receptor.

11. The method of claim 8, wherein said antagonist of the A_{2B} adenosine receptor is selected from the group consisting of theophylline, PSB36, SCH442416, MRS1754 and MRS37

12. A method of treating or preventing sickle cell disease symptoms in a mammal, comprising:
administering to a person such symptoms a composition comprising an effective amount of at least one inhibitor of adenosine signaling and a pharmaceutically-acceptable carrier, wherein the inhibitor of adenosine signaling has at least one activity selected from the group consisting of decreasing adenosine levels in the mammal, inhibiting adenosine receptor activity in the mammal, and inhibiting signaling pathways downstream of an adenosine receptor in the mammal.

13. The method of claim 12, wherein said symptoms selected from the group consisting of sickling of erythrocytes, oxygen release, increased hemoglobin (HbS) polymerization, hemolysis, tissue congestion and organ damage.

14. The method of claim 12, wherein said symptom is sickling of erythrocytes.
